(19) United States
(12) Patent Application Publication
(10) Pub. No.: US 2022/0379287 A1
(43) Pub. Date: Dec. 1, 2022

(54) DEHYDROGENATION CATALYST

(71) Applicant: NATIONAL UNIVERSITY CORPORATION HOKKAIDO UNIVERSITY, Sapporo-shi (JP)

(72) Inventors: Shinya FURUKAWA, Sapporo-shi (JP); Yuki NAKAYA, Sapporo-shi (JP); Eigo HAYASHIDA, Sapporo-shi (JP)

(21) Appl. No.: 17/690,421

(22) Filed: Mar. 9, 2022

(30) Foreign Application Priority Data
May 28, 2021 (JP) 2021-090373
Oct. 8, 2021 (JP) 2021-166497

(51) Int. Cl. B01J 23/62; B01J 21/08; B01J 23/89; C07C 5/333

(52) U.S. Cl. CPC B01J 23/628; B01J 21/08; B01J 23/62; B01J 23/8966; B01J 23/896; B01J 23/8946; C07C 5/3337; C07C 2523/62; C07C 2521/08; C07C 2523/89

(57) ABSTRACT

A dehydrogenation catalyst for producing propylene by a dehydrogenation reaction of propane, the dehydrogenation catalyst including a platinum element and an element M1 and may contain an element M2 as active components, wherein the element M1 is one or more elements selected from the group consisting of a gallium element, a cobalt element, a copper element, a germanium element, a tin element and an iron element, the element M2 is one or more elements selected from the group consisting of a lead element and a calcium element, and the platinum element and the element M1 form an alloy.

### BACKGROUND OF THE INVENTION

### Field of the Invention

The present invention relates to a catalyst for dehydrogenation (dehydrogenation catalyst).

Priority is claimed on Japanese Patent Application No. 2021-090373, filed May 28, 2021 and Japanese Patent Application No. 2021-166497, filed Oct. 8, 2021, the contents of which are incorporated herein by reference.

### Description of the Related Art

Propylene is a basic chemical product for producing various chemical products such as resins, surfactants, dyes, and medicines. In recent years, the supply of propylene has decreased due to the shift in raw materials for steam crackers from crude oil-derived naphtha to shale gas-derived ethane.

Because of such backgrounds, the production of propylene by a dehydrogenation reaction of propane has been attracting attention. Since the dehydrogenation reaction of propane is an endothermic reaction, a high temperature of 600° C. or higher is required for the reaction to proceed. Intensive and extensive studies have been conducted so far on catalysts for dehydrogenation of propane, and a catalyst containing a platinum metal is known. However, when the dehydrogenation reaction of propane is carried out at 600° C. or higher using a conventional catalyst containing a platinum metal, the activity is reduced due to the deposition of coke on the catalyst and/or the sintering of the active metal including a platinum metal.

Non-Patent Document 1 discloses a catalyst containing a platinum-tin alloy. It is disclosed that by using a catalyst containing a platinum-tin alloy, a decrease in activity is suppressed in a dehydrogenation reaction of propane at 600° C. or higher as compared with a conventional catalyst containing a platinum metal.

### PRIOR ART DOCUMENTS

### Non-Patent Document

[Non-Patent Document 1] J. Feng et al., Chin. J. Chem. Eng., 2014, 22, 1232.

### SUMMARY OF THE INVENTION

### Problems to be Solved by the Invention

However, the catalyst life of the propane dehydrogenation catalyst described in Non-Patent Document 1 is not sufficient. The present invention has been made in view of the above circumstances, with an object of providing a dehydrogenation catalyst for producing propylene by a dehydrogenation reaction of propane, which has a longer life than that of a conventional catalyst.

### Means for Solving the Problem

In order to solve the above problems, the present invention includes the following aspects.

[1] A dehydrogenation catalyst for producing propylene by a dehydrogenation reaction of propane, the dehydrogenation catalyst including a platinum element, a gallium element and an element M as active components, wherein the aforementioned element M is one or more elements selected from the group consisting of a lead element, a calcium element, a cobalt element, a copper element, a germanium element and a tin element.

[2] The dehydrogenation catalyst according to [1], which contains a lead element as the aforementioned element M.

[3] The dehydrogenation catalyst according to [1], which contains a cobalt element, a copper element, a germanium element and a tin element as the aforementioned element M.

[4] The dehydrogenation catalyst according to any one of [1] to [3], which contains a calcium element as the aforementioned element M.

[5] The dehydrogenation catalyst according to any one of [1] to [4], wherein the aforementioned platinum element and the aforementioned gallium element form an alloy, and the aforementioned element M is present on a surface of the aforementioned alloy.

[6] The dehydrogenation catalyst according to any one of [1] to [5], wherein the aforementioned platinum element, the aforementioned gallium element and the aforementioned element M form an alloy.

[7] The dehydrogenation catalyst according to any one of [1] to [6], wherein the aforementioned active component is supported on a silica carrier.

In order to solve the above problems, the present invention also includes the following aspects.

[8] A dehydrogenation catalyst for producing propylene by a dehydrogenation reaction of propane, the dehydrogenation catalyst including a platinum element and an element M1 and may contain an element M2 as active components, wherein the aforementioned element M1 is one or more elements selected from the group consisting of a gallium element, a cobalt element, a copper element, a germanium element, a tin element and an iron element, the aforementioned element M2 is one or more elements selected from the group consisting of a lead element and a calcium element, and the aforementioned platinum element and the aforementioned element M1 form an alloy (provided that a dehydrogenation catalyst containing only a tin element as the element M1 and not containing an element M2, a dehydrogenation catalyst containing only a gallium element as the element M1 and not containing an element M2, a dehydrogenation catalyst containing only a cobalt element as the element M1 and not containing an element M2, a dehydrogenation catalyst containing only a copper element as the element M1 and not containing an element M2, a dehydrogenation catalyst containing only a germanium element as the element M1 and not containing an element M2, and a dehydrogenation catalyst containing only a germanium element as the element M1 and containing only a calcium element as the element M2 are excluded).

[9] The dehydrogenation catalyst according to [8], which contains a gallium element as the aforementioned element M1.

[10] The dehydrogenation catalyst according to [9], which contains a lead element as the aforementioned element M2.

[11] The dehydrogenation catalyst according to [10], wherein the aforementioned lead element is present as an atom on a surface of the aforementioned alloy.

[12] The dehydrogenation catalyst according to [9], which contains a cobalt element, a copper element, a germanium element and a tin element as the aforementioned element M1.

[13] The dehydrogenation catalyst according to [9], which contains a cobalt element, a copper element and an iron element as the aforementioned element M1.

[14] The dehydrogenation catalyst according to [8], which contains a copper element as the aforementioned element M1.

[15] The dehydrogenation catalyst according to any one of [8] to [14], which contains a calcium element as the aforementioned element M2.

[16] The dehydrogenation catalyst according to any one of [8] to [15], wherein the aforementioned active component is supported on a silica carrier.

[17] The dehydrogenation catalyst according to [14], which contains a cobalt element and a gallium element as the aforementioned element M1.

[18] The dehydrogenation catalyst according to [17], which contains one or more elements selected from the group consisting of a germanium element, a tin element and an iron element as the aforementioned element M1.

[19] The dehydrogenation catalyst according to [17] or [18], which contains a calcium element as the aforementioned element M2.

[20] The dehydrogenation catalyst according to any one of [17] to [19], wherein the aforementioned active component is supported on a silica carrier.

### Effects of the Invention

According to the present invention, it is possible to provide a dehydrogenation catalyst for producing propylene by a dehydrogenation reaction of propane, which has a longer life than that of a conventional catalyst.

### BRIEF DESCRIPTION OF THE DRAWINGS

FIG. 1 is a diagram showing a crystal structure (unit cell) of a platinum-gallium alloy.

FIG. 2 is a diagram showing an atomic arrangement of a surface ((111) plane) of a platinum-gallium alloy.

FIG. 3 is a diagram showing a putative mechanism in which lead atoms selectively cover Pt₃ sites on a surface of a platinum-gallium alloy.

FIG. 4 is a diagram showing a crystal structure (unit cell) of a platinum-gallium-cobalt-copper-germanium-tin alloy.

FIG. 5 is a diagram showing the results of dehydrogenation reactions of propane using the catalysts of Example 1 and Comparative Examples 1 to 7.

FIG. 6 is a diagram showing the results of dehydrogenation reactions of propane using the catalysts of Examples 1 to 4 and Comparative Example 3.

FIG. 7 is a diagram showing the results of dehydrogenation reactions of propane using the catalysts of Examples 5 to 7 and Comparative Example 3.

FIG. 8 is a diagram showing the results of dehydrogenation reactions of propane using the catalysts of Examples 5 and 7.

FIG. 9 is a diagram showing the results of dehydrogenation reactions of propane using the catalysts of Examples 6, 8 and 9 and Comparative Example 3.

FIG. 10 is a diagram showing the results of dehydrogenation reactions of propane using the catalysts of Comparative Examples 3 and 8 to 11.

FIG. 11 is a diagram showing the results of dehydrogenation reactions of propane using the catalysts of Example 6, and Comparative Examples 3, 12 and 13.

FIG. 12 is a diagram showing the results of dehydrogenation reactions of propane using the catalysts of Comparative Examples 3 and 14 to 18.

FIG. 13 is a diagram showing the results of dehydrogenation reactions of propane using the catalysts of Example 6 and Comparative Examples 19 to 21.

FIG. 14 is a diagram showing the results of dehydrogenation reactions of propane using the catalysts of Example 6 and Comparative Examples 22 to 24.

FIG. 15 is a diagram showing the results of dehydrogenation reactions of propane using the catalysts of Example 10 and Comparative Example 25.

FIG. 16 is a diagram showing the results of a dehydrogenation reactions of propane using the catalysts of Examples 5 and 10.

FIG. 17 is a diagram showing the results of a dehydrogenation reaction of propane using the catalyst of Example 10.

FIG. 18 is a diagram showing the results of dehydrogenation reactions of propane using the catalysts of Examples 10 to 12 and Comparative Example 25.

### DETAILED DESCRIPTION OF THE INVENTION

Hereinafter, embodiments of the present invention will be described in detail, but the following description is an example of the embodiments of the present invention, and the present invention is not limited to these contents, and can be modified and implemented within the scope of the gist thereof.

<<Dehydrogenation catalyst>>

A first dehydrogenation catalyst of the present invention is a dehydrogenation catalyst for producing propylene by a dehydrogenation reaction of propane. The dehydrogenation catalyst contains a platinum element, a gallium element and an element M as active components, and the aforementioned element M is one or more elements selected from the group consisting of a lead element, a calcium element, a cobalt element, a copper element, a germanium element and a tin element. The element M may consist only of one type of element, or may contain two or more types of elements.

A second dehydrogenation catalyst of the present invention contains a platinum element and an element M1, may contain an element M2 as active components, and the aforementioned element M1 is one or more elements selected from the group consisting of a gallium element, a cobalt element, a copper element, a germanium element, a tin element and an iron element, and the aforementioned element M2 is one or more elements selected from the group consisting of a lead element and a calcium element. The aforementioned platinum element and the element M1 form an alloy. The element M1 may consist only of one type of element, or may contain two or more types of elements. The element M2 may consist only of one type of element, or may contain two or more types of elements. The expression “may contain an element M2” means that the element M2 may or may not be contained.

However, the second dehydrogenation catalyst does not include a dehydrogenation catalyst containing only a tin element as the element M1 and not containing an element M2, a dehydrogenation catalyst containing only a gallium element as the element M1 and not containing an element M2, a dehydrogenation catalyst containing only a cobalt element as the element M1 and not containing an element M2. a dehydrogenation catalyst containing only a copper element as the element M1 and not containing an element M2, a dehydrogenation catalyst containing only a germanium element as the element M1 and not containing an element M2, and a dehydrogenation catalyst containing only a germanium element as the element M1 and containing only a calcium element as the element M2.

The sum of the types of elements M1 and elements M2 contained in the second dehydrogenation catalyst is preferably from 2 to 8, and more preferably from 2 to 6.

In an embodiment of the first dehydrogenation catalyst, it is preferable that the platinum element and the gallium element serving as the aforementioned active components form an alloy. In an embodiment of the second dehydrogenation catalyst, it is preferable to contain a gallium element as the element M1. That is, the aforementioned active component preferably contains a platinum-gallium alloy. In the case of the first dehydrogenation catalyst, the element M is preferably present on the surface of the platinum-gallium alloy. The form of the element M present on the surface of the platinum-gallium alloy is preferably an atom. The form of the element M that is not present on the surface of the platinum-gallium alloy may be a metal or an oxide. When two or more types of elements M are contained, a part or all of the elements M may form an alloy.

In an embodiment of the first dehydrogenation catalyst, it is preferable that the platinum element, the gallium element and the element M serving as the aforementioned active components form an alloy. That is, the aforementioned active component preferably contains a platinum-gallium-M alloy. When two or more types of elements M are contained, the platinum element, the gallium element and all of the elements M may form an alloy, or the platinum element, the gallium element and a part of the elements M may form an alloy. In an embodiment of the second dehydrogenation catalyst, it is preferable to contain a gallium element and (an)other element(s) as the elements M1.

In an embodiment of the first dehydrogenation catalyst and the second dehydrogenation catalyst, the active component is preferably supported on a silica carrier. By supporting the active component on the silica carrier, the number of active sites of the active component on the surface of the dehydrogenation catalyst can be increased. As the silica carrier, a silica carrier known in the art can be used. The silica carrier is preferably a porous silica carrier.

In the first dehydrogenation catalyst and the second dehydrogenation catalyst, the content ratio of the aforementioned active component with respect to the total mass of the dehydrogenation catalyst is preferably from 0.25 to 21% by mass, more preferably from 2 to 16% by mass, and still more preferably from 2 to 11% by mass. When the content ratio of the active component is equal to or greater than the lower limit value of the above range, the propylene selectivity and the catalyst life improve. When the content ratio of the active component is equal to or less than the upper limit value of the above range, the catalytic activity improves.

In the first dehydrogenation catalyst and the second dehydrogenation catalyst, the content ratio of the platinum element with respect to the total mass of the dehydrogenation catalyst is preferably from 0.1 to 10% by mass, more preferably from 0.5 to 5% by mass, and still more preferably from 0.5 to 3% by mass. When the content ratio of the platinum element is equal to or greater than the lower limit value of the above range, the catalytic activity improves. When the content ratio of the platinum element is equal to or less than the upper limit value of the above range, the propylene selectivity improves.

In the first dehydrogenation catalyst and the second dehydrogenation catalyst containing gallium as the element M1, the content ratio of the gallium element with respect to the total mass of the dehydrogenation catalyst is preferably from 0.05 to 6% by mass, more preferably from 0.5 to 7% by mass, and still more preferably from 0.5 to 5% by mass. When the content ratio of the gallium element is equal to or greater than the lower limit value of the above range, the propylene selectivity improves. When the content ratio of the gallium element is equal to or less than the upper limit value of the above range, the catalytic activity improves.

In the first dehydrogenation catalyst, the content ratio of the element M with respect to the total mass of the dehydrogenation catalyst is preferably from 0.1 to 5% by mass, more preferably from 1 to 4% by mass, and still more preferably from 1 to 3% by mass. When the content ratio of the element M is equal to or greater than the lower limit value of the above range, the propylene selectivity improves. When the content ratio of the element M is equal to or less than the upper limit value of the above range, the catalytic activity improves.

In the present specification, the content ratios of “platinum element, gallium element, element M, element M1 and element M2” can be measured by inductively coupled plasma emission spectrometry (ICP). For example, after dissolving the dehydrogenation catalyst in hydrochloric acid, the amount of each metal can be measured using an inductively coupled plasma emission spectrometer.

Physical property values of the first dehydrogenation catalyst and the second dehydrogenation catalyst are preferably in the following ranges.

A BET specific surface area of the first dehydrogenation catalyst and the second dehydrogenation catalyst due to nitrogen adsorption is preferably from 300 to 900 m²/g, more preferably from 400 to 900 m²/g, and still more preferably from 500 to 900 m²/g. When the specific surface area of the dehydrogenation catalyst is equal to or greater than the lower limit value of the above range, the catalytic activity improves.

In the present specification, the “BET specific surface area” can be measured by nitrogen adsorption measurement.

Hereinafter, a preferred embodiment of the combination of elements M in the first dehydrogenation catalyst will be described with reference to an example. Further, a preferred embodiment of the combination of the element M1 and the element M2 in the second dehydrogenation catalyst will be described with reference to an example in a similar manner. It should be noted that the preferred range of the physical property values, the type of the preferred carrier, and the like of the above-mentioned catalyst can also be applied to those of dehydrogenation catalysts in the following first embodiment, 20 second embodiment, embodiment 2-1, embodiment 2-2 and third embodiment.

### First Embodiment

In the case of the first dehydrogenation catalyst, the active component of the dehydrogenation catalyst of the first embodiment (hereinafter, also referred to as “dehydrogenation catalyst 1”) contains a platinum element, a gallium element, and a lead element serving as the element M. The element M may contain an element other than the lead element, but the element M is preferably composed only of the lead element. In the case of the second dehydrogenation catalyst, the active component of the dehydrogenation catalyst 1 contains a platinum element, a gallium element as an element M1, and a lead element as an element M2. The element M1 may contain an element other than the gallium element, but the element M1 is preferably composed only of the gallium element. The element M2 may contain an element other than the lead element, but the element M2 is preferably composed only of the lead element.

In the active component of the dehydrogenation catalyst 1, it is preferable that the platinum element and the gallium element form an alloy. That is, the aforementioned active component preferably contains a platinum-gallium alloy. The lead element is preferably present on the surface of the platinum-gallium alloy. That is, the aforementioned active component preferably contains a complex in which a lead element (lead atom) is present on the surface of a platinum-gallium alloy. The form of the lead element that is not present on the surface of the platinum-gallium alloy may be a metal or an oxide. When the first dehydrogenation catalyst contains an element M other than the lead element, a part or all of the lead element may form an alloy with the element M other than the lead element. In the second dehydrogenation catalyst, a part or all of the lead element may form an alloy with the element M1.

The content ratio of the aforementioned active component with respect to the total mass of the dehydrogenation catalyst 1 is preferably from 0.25 to 21% by mass, more preferably from 2 to 16% by mass, and still more preferably from 2 to 11% by mass. When the content ratio of the active component is equal to or greater than the lower limit value of the above range, the catalyst life improves. When the content ratio of the active component is equal to or less than the upper limit value of the above range, the catalytic activity improves.

The content ratio of the platinum element with respect to the total mass of the dehydrogenation catalyst 1 is preferably from 0.1 to 10% by mass, more preferably from 0.5 to 5% by mass, and still more preferably from 0.5 to 3% by mass. When the content ratio of the platinum element is equal to or greater than the lower limit value of the above range, the catalytic activity improves. When the content ratio of the platinum element is equal to or less than the upper limit value of the above range, the propylene selectivity improves.

The content ratio of the gallium element with respect to the total mass of the dehydrogenation catalyst 1 is preferably from 0.05 to 6% by mass, more preferably from 0.5 to 7% by mass, and still more preferably from 0.5 to 5% by mass. When the content ratio of the gallium element is equal to or greater than the lower limit value of the above range, the propylene selectivity improves. When the content ratio of the gallium element is equal to or less than the upper limit value of the above range, the catalytic activity improves.

The content ratio of the lead element with respect to the total mass of the dehydrogenation catalyst 1 is preferably from 0.1 to 5% by mass, more preferably from 1 to 4% by mass, and still more preferably from 1 to 3% by mass. When the content ratio of the lead element is equal to or greater than the lower limit value of the above range, the propylene selectivity improves. When the content ratio of the lead element is equal to or less than the upper limit value of the above range, the catalytic activity improves.

A molar ratio of the platinum element with respect to the gallium element (Pt/Ga) in the dehydrogenation catalyst 1 is preferably from 0.3 to 1, more preferably from 0.5 to 1, and still more preferably from 0.7 to 1. When the Pt/Ga ratio is equal to or greater than the lower limit value of the above range, the propylene selectivity improves. When the Pt/Ga ratio is equal to or less than the upper limit value of the above range, the propylene selectivity improves.

A molar ratio of the platinum element with respect to the lead element (Pt/Pb) in the dehydrogenation catalyst 1 is preferably from 1.2 to 5.0, more preferably from 1.2 to 2.5, still more preferably from 1.3 to 2, and particularly preferably from 1.5 to 2. When the Pt/Pb ratio is equal to or greater than the lower limit value of the above range, the catalyst life improves. When the Pt/Pb ratio is equal to or less than the upper limit value of the above range, the catalytic activity improves.

A molar ratio of the lead element with respect to the total of platinum element and gallium element (Pb/(Pt+Ga)) in the dehydrogenation catalyst 1 is preferably from 0.2 to 0.6, more preferably from 0.3 to 0.6, and still more preferably from 0.3 to 0.5. When the Pb/(Pt+Ga) ratio is equal to or greater than the lower limit value of the above range, the catalyst life improves. When the Pb/(Pt+Ga) ratio is equal to or less than the upper limit value of the above range, the catalytic activity improves.

An average particle size of the aforementioned complex serving as the active component of the dehydrogenation catalyst 1 is preferably from 0.2 to 4 nm, more preferably from 0.2 to 3 nm, and still more preferably from 0.2 to 2.8 nm. When the average particle size of the complex is equal to or greater than the lower limit value of the above range, the catalyst life improves. When the average particle size of the complex is equal to or less than the upper limit value of the above range, the catalytic activity improves. The average particle size of the complex can be measured by a scanning transmission electron microscope (STEM). A specific method for measuring the average particle size of the complex will be explained in Examples described later. For example, it is possible to observe the longest diameters of 100 particles (active components) and to take an average of these values as the average particle size.

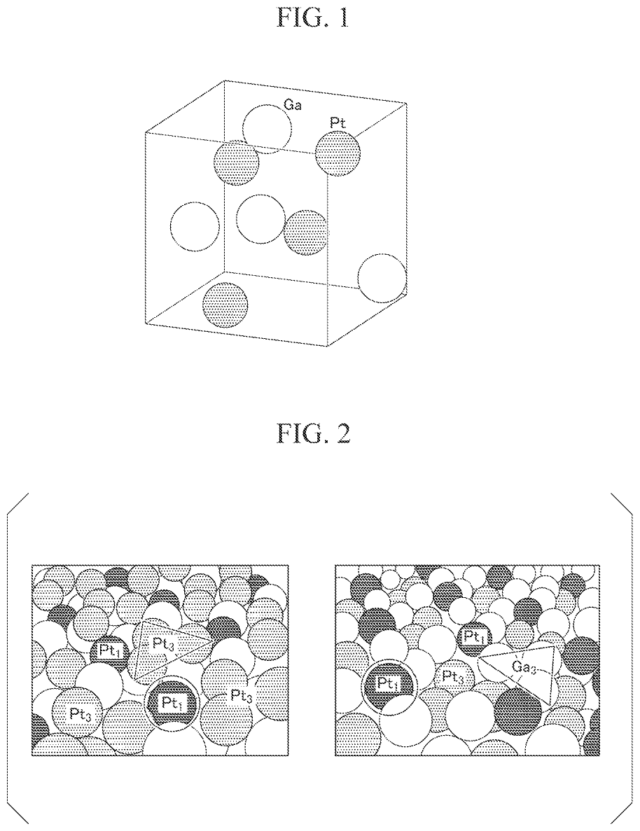

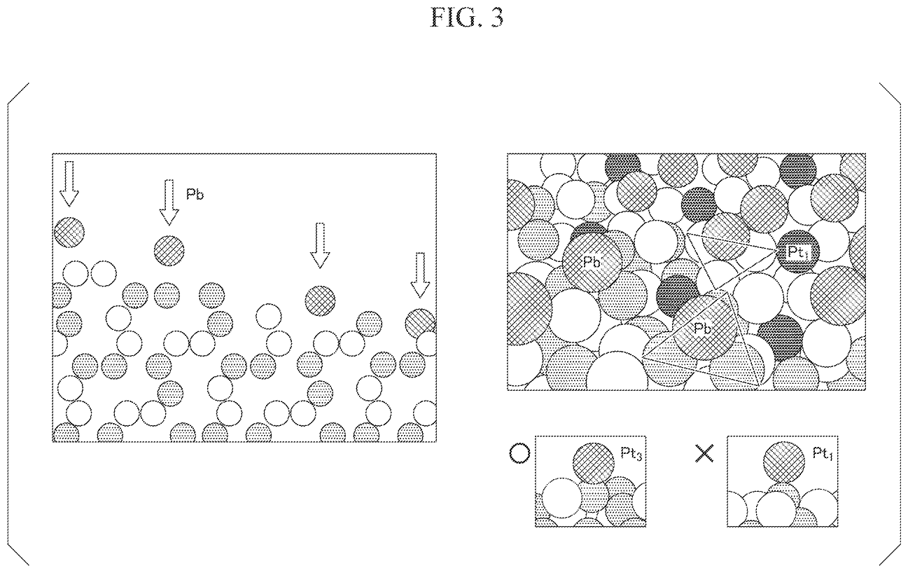

It is known that the crystal structure of platinum-gallium alloys belongs to a space group (P2₁3). FIG. 1 shows a crystal structure (unit cell) of a platinum-gallium alloy. An atomic arrangement of the surface ((111) plane) of the platinum-gallium alloy is shown in FIG. 2. On the surface of the platinum-gallium alloy, a site where three platinum atoms are in contact with each other (hereinafter, also referred to as “Pt₃ site”) and a site where one platinum atom is surrounded by three gallium atoms (hereinafter, also referred to as “Pt₁ site”) are present. In FIG. 2, the white circles represent Ga atoms, the dark black circles represent Pt atoms at the Pt₁ site, and the light black circles represent Pt atoms at the Pt₃ site (the same applies to FIG. 3).

As a result of the intensive studies by the inventors of the present application, it was found that not only the dehydrogenation reaction of propane but also the side reaction of producing coke occurs at the Pt₃ site, while substantially only the dehydrogenation reaction of propane proceeds at the Pt₁ site. As described above, the activity of the dehydrogenation catalyst is reduced by the deposition of coke derived from the aforementioned side reaction. That is, if the side reaction derived from the Pt₃ site can be suppressed, it is presumed that the decrease in the activity of the dehydrogenation catalyst is suppressed.

A ratio of the number of Pt₁ sites and the number of Pt₃ sites is determined by the crystal structure. Therefore, it is difficult to control the number of Pt₁ sites and the number of Pt₃ sites on the surface of the platinum-gallium alloy. The inventors of the present application investigated whether the side reaction at the Pt₃ site could be suppressed by covering the Pt₃ site with an atom other than platinum and gallium atoms. As a result, the inventors of the present application have found that when a lead atom is used, only the Pt₃ site is covered while the Pt₁ site is not covered, and the side reaction at the Pt₃ site is suppressed.

FIG. 3 is a diagram showing a putative mechanism in which lead atoms selectively cover the Pt₃ sites on the surface of a platinum-gallium alloy. As described above, the Pt₃ site is a site in which three platinum atoms are in contact with each other, and the Pt₁ site is a site in which one platinum atom is surrounded by three gallium atoms. As shown in FIG. 3, it is presumed that the lead atom can stably cover the Pt₃ site by being located in a gap between the three platinum atoms in the Pt₃ site. On the other hand, since the Pt₁ site contains one platinum atom, it is presumed that the lead atom cannot cover the Pt₁ site from the viewpoint of stability. It is presumed that the lead atom can selectively cover only the Pt₃ site because of the above mechanism.

The inventors of the present application have also conducted similar studies on elements other than lead atoms (for example, an indium element and a tin element), but it was found that lead was the only element capable of selectively covering the Pt₃ sites.

The covering of the Pt₃ site by the lead atoms can be confirmed by infrared (IR) absorption spectrometry of adsorbed CO. More specifically, in a case in which IR is measured in a state where carbon monoxide is adsorbed on the dehydrogenation catalyst 1, when a ratio of the peak intensity at 2080 cm⁻¹ with respect to the peak intensity at 2040 cm⁻¹ is 0.1 or less, it can be determined that the lead atom covers the Pt₃ site. In the IR spectroscopy of CO adsorption, the peak at 2040 cm⁻¹ is a peak attributed to the Pt₁ site, and the peak at 2080 cm⁻¹ is a peak attributed to the Pt₃ site. Although the lower limit value of the above ratio is not particularly limited, it is, for example, 0.0001 or higher. That is, the above ratio is preferably from 0.0001 to 0.1.

The dispersity of platinum elements measured by CO adsorption on the dehydrogenation catalyst 1 is preferably from 1 to 10%, more preferably from 2 to 8%, still more preferably from 3 to 7%, and particularly preferably from 3 to 6%. When the lead atom covers the Pt₃ site, the dispersity of platinum elements decreases. The method for measuring CO adsorption and the method for calculating the dispersity of platinum elements will be described in detail in Examples.

The presence of lead atoms on the surface of the platinum-gallium alloy can be confirmed by XPS (X-ray photoelectron spectroscopy) measurement. If lead atoms are present on the surface of the platinum-gallium alloy, an increase in binding energy (eV) derived from Pt4f is observed when the dehydrogenation catalyst 1 is sputtered with Ar⁺. The increase in binding energy (eV) derived from Pt4f is caused by the disappearance of electron donation from Pb to Pt due to the removal of lead atoms by sputtering. In the present embodiment, a value obtained by dividing the binding energy (eV) derived from Pt4f of the dehydrogenation catalyst 1 before sputtering with the binding energy (eV) derived from Pt4f of the dehydrogenation catalyst 1 after 0.5 nm sputtering is preferably 0.3 or less. Although the lower limit value of the above ratio is not particularly limited, it is, for example, 0.0001 or higher. That is, the above ratio is preferably from 0.001 to 0.3.

### Second Embodiment

In the case of the second dehydrogenation catalyst, the active component of the dehydrogenation catalyst of the second embodiment (hereinafter, also referred to as “dehydrogenation catalyst 2”) contains a platinum element, and a gallium element, a cobalt element and a copper element serving as the elements M1. The element M1 may contain an element other than the above-mentioned elements, but the element M1 may be composed only of the above-mentioned elements. When the element M1 contains an element other than the above-mentioned elements (additional element M1), the additional element M1 is preferably one or more elements selected from the group consisting of a germanium element, a tin element and an iron element. Further, an element M2 may be contained. The platinum element and a plurality of the elements M1 form an alloy.

As for the preferred range of the content ratio of the active component with respect to the total mass of the dehydrogenation catalyst 2 and the preferred ranges of the content ratios of each of the platinum element, the gallium element, the cobalt element and the copper element, the ranges described for the dehydrogenation catalysts 2-1 and 2-2 described below can be applied. Further, when the dehydrogenation catalyst 2 contains the aforementioned additional element M1, as for the preferred ranges of the respective content ratios of the germanium element, the tin element and the iron element with respect to the total mass of the dehydrogenation catalyst 2, the ranges described for the dehydrogenation catalyst 2-1 or 2-2 described later can be applied.

As for the preferred range of a ratio of the total number of moles of the typical metals with respect to the total number of moles of the transition metals contained in the dehydrogenation catalyst 2, the same range as that of a ratio of ((typical metal group 1)/(transition metal group 1)) of the dehydrogenation catalyst 2-1 described later can be applied.

As for the preferred ranges of a ratio of the number of moles of the platinum element, a ratio of the number of moles of the cobalt element, a ratio of the number of moles of the copper element, and a ratio of the number of moles of the gallium element with respect to the total number of moles of the transition metals contained in the dehydrogenation catalyst 2, the ranges described for ratios of (Pt/(transition metal group 1)), (Co/(transition metal group 1)), (Cu/(transition metal group 1)) and (Ga/(typical metal group 1)) of the dehydrogenation catalyst 2-1 described later can be applied, respectively. Further, ranges described for ratios of (Pt/(transition metal group 2)), (Co/(transition metal group 2)), (Cu/(transition metal group 2)) and (Ga/(typical metal group 2)) of the dehydrogenation catalyst 2-2 described later may be applied.

Moreover, when the dehydrogenation catalyst 2 contains the aforementioned additional element M1, as for the preferred ranges of a ratio of the number of moles of the germanium element and a ratio of the number of moles of the tin element with respect to the total number of moles of the typical metals contained in the dehydrogenation catalyst 2, the ranges described for ratios of (Ge/(typical metal group 1)) and (Sn/(typical metal group 1)) of the dehydrogenation catalyst 2-1 described later can be applied, respectively. Furthermore, as for the preferred range of a ratio of the number of moles of the iron element with respect to the total number of moles of the transition metals contained in the dehydrogenation catalyst 2, the range described for a ratio of (Fe/(transition metal group 2)) of the dehydrogenation catalyst 2-2 described later can be applied.

As for the preferred range of the average particle size of the alloy formed by the platinum element and a plurality of elements M1, ranges described for a six element alloy of the dehydrogenation catalyst 2-1 and a five element alloy of the dehydrogenation catalyst 2-2 described later can be applied.

### Embodiment 2-1

In the case of the first dehydrogenation catalyst, the active component of the dehydrogenation catalyst of the embodiment 2-1 (hereinafter, also referred to as “dehydrogenation catalyst 2-1”) contains a platinum element, a gallium element, and a cobalt element, a copper element, a germanium element and a tin element serving as the elements M. The element M may contain an element other than the above-mentioned elements, but the element M is preferably composed only of the above-mentioned elements. In the case of the second dehydrogenation catalyst, the active component of the dehydrogenation catalyst 2-1 contains a platinum element, and a gallium element, a cobalt element, a copper element, a germanium element and a tin element serving as the elements M1. The element M1 may contain an element other than the above-mentioned elements, but the element M1 is preferably composed only of the above-mentioned elements. Further, an element M2 may be contained.

In the active component of the dehydrogenation catalyst 2-1, it is preferable that the platinum element, the gallium element, the cobalt element, the copper element, the germanium element and the tin element form an alloy. That is, the aforementioned active component preferably contains a platinum-gallium-cobalt-copper-germanium-tin alloy (hereinafter, also referred to as “six element alloy”).

The content ratio of the aforementioned active component with respect to the total mass of the dehydrogenation catalyst 2-1 is preferably from 0.3 to 28% by mass, more preferably from 0.3 to 18% by mass, and still more preferably from 0.3 to 11.5% by mass. When the content ratio of the active component is equal to or greater than the lower limit value of the above range, the catalyst life improves. When the content ratio of the active component is equal to or less than the upper limit value of the above range, the catalyst life improves.

The content ratio of the platinum element with respect to the total mass of the dehydrogenation catalyst 2-1 is preferably from 0.1 to 5% by mass, more preferably from 0.1 to 3% by mass, and still more preferably from 0.1 to 1% by mass. When the content ratio of the platinum element is equal to or greater than the lower limit value of the above range, the catalyst life improves. When the content ratio of the platinum element is equal to or less than the upper limit value of the above range, the catalyst life improves.

The content ratio of the gallium element with respect to the total mass of the dehydrogenation catalyst 2-1 is preferably from 0.05 to 5% by mass, more preferably from 0.05 to 3% by mass, and still more preferably from 0.05 to 2% by mass. When the content ratio of the gallium element is equal to or greater than the lower limit value of the above range, the catalyst life improves. When the content ratio of the gallium element is equal to or less than the upper limit value of the above range, the catalytic activity improves.

The content ratio of the cobalt element with respect to the total mass of the dehydrogenation catalyst 2-1 is preferably from 0.03 to 3% by mass, more preferably from 0.03 to 2% by mass, and still more preferably from 0.03 to 1.5% by mass. When the content ratio of the cobalt element is equal to or greater than the lower limit value of the above range, the catalyst life improves. When the content ratio of the cobalt element is equal to or less than the upper limit value of the above range, the catalytic activity improves.

The content ratio of the copper element with respect to the total mass of the dehydrogenation catalyst 2-1 is preferably from 0.03 to 4% by mass, more preferably from 0.03 to 3% by mass, and still more preferably from 0.03 to 2% by mass. When the content ratio of the copper element is equal to or greater than the lower limit value of the above range, the catalyst life improves. When the content ratio of the copper element is equal to or less than the upper limit value of the above range, the catalytic activity improves.

The content ratio of the germanium element with respect to the total mass of the dehydrogenation catalyst 2-1 is preferably from 0.03 to 4% by mass, more preferably from 0.03 to 3% by mass, and still more preferably from 0.03 to 2% by mass. When the content ratio of the germanium element is equal to or greater than the lower limit value of the above range, the catalyst life improves. When the content ratio of the germanium element is equal to or less than the upper limit value of the above range, the catalytic activity improves.

The content ratio of the tin element with respect to the total mass of the dehydrogenation catalyst 2-1 is preferably from 0.06 to 7% by mass, more preferably from 0.06 to 4% by mass, and still more preferably from 0.06 to 3% by mass. When the content ratio of the tin element is equal to or greater than the lower limit value of the above range, the catalyst life improves. When the content ratio of the tin element is equal to or less than the upper limit value of the above range, the catalytic activity improves.

When the inventors of the present invention analyzed the crystal structure of the six element alloy, it was found to have the same FeAs crystal structure as that of the platinum-germanium alloy. Further intensive studies by the inventors of the present invention revealed that platinum, cobalt and copper are located at the platinum site, and gallium, germanium and tin are located at the germanium site in the platinum-germanium alloy. Hereinafter, platinum, cobalt and copper are collectively referred to as “transition metal group 1”, and gallium, germanium and tin are collectively referred to as “typical metal group 1”.

The ratio of the total number of moles of the typical metal group 1 with respect to the total number of moles of the transition metal group 1 ((typical metal group 1)/(transition metal group 1)) is preferably from 0.8 to 1.5, more preferably from 0.9 to 1.3, and still more preferably from 1 to 1.2. When the ((typical metal group 1)/(transition metal group 1)) ratio is equal to or greater than the lower limit value of the above range, the catalyst life improves. When the ((typical metal group 1)/(transition metal group 1)) ratio is equal to or less than the upper limit value of the above range, the catalytic activity improves.

The ratio of the number of moles of the platinum element with respect to the total number of moles of the transition metal group 1 (Pt/(transition metal group 1)) is preferably from 0.1 to 0.5, more preferably from 0.2 to 0.5, still more preferably from 0.2 to 0.4, and particularly preferably from 0.2 to 0.35. When the (Pt/(transition metal group 1)) ratio is equal to or greater than the lower limit value of the above range, the catalytic activity improves. When the (Pt/(transition metal group 1)) ratio is equal to or less than the upper limit value of the above range, the catalyst life improves.

The ratio of the number of moles of the cobalt element with respect to the total number of moles of the transition metal group 1 (Co/(transition metal group 1)) is preferably from 0.25 to 0.45, more preferably from 0.30 to 0.45, and still more preferably from 0.30 to 0.40. When the (Co/(transition metal group 1)) ratio is equal to or greater than the lower limit value of the above range, the catalyst life improves. When the (Co/(transition metal group 1)) ratio is equal to or less than the upper limit value of the above range, the catalytic activity improves.

The ratio of the number of moles of the copper element with respect to the total number of moles of the transition metal group 1 (Cu/(transition metal group 1)) is preferably from 0.25 to 0.45, more preferably from 0.30 to 0.45, and still more preferably from 0.30 to 0.40. When the (Cu/(transition metal group 1)) ratio is equal to or greater than the lower limit value of the above range, the catalyst life improves. When the (Cu/(transition metal group 1)) ratio is equal to or less than the upper limit value of the above range, the catalytic activity improves.

The ratio of the number of moles of the gallium element with respect to the total number of moles of the typical metal group 1 (Ga/(typical metal group 1)) is preferably from 0.30 to 0.50, more preferably from 0.35 to 0.50, and still more preferably from 0.35 to 0.45. When the (Ga/(typical metal group 1)) ratio is equal to or greater than the lower limit value of the above range, the catalyst life improves. When the (Ga/(typical metal group 1)) ratio is equal to or less than the upper limit value of the above range, the catalytic activity improves.

The ratio of the number of moles of the germanium element with respect to the total number of moles of the typical metal group 1 (Ge/(typical metal group 1)) is preferably from 0.20 to 0.40, more preferably from 0.25 to 0.40, and still more preferably from 0.20 to 0.40. When the (Ge/(typical metal group 1)) ratio 1 is equal to or greater than the lower limit value of the above range, the catalyst life improves. When the (Ge/(typical metal group 1)) ratio is equal to or less than the upper limit value of the above range, the catalytic activity improves.

The ratio of the number of moles of the tin element with respect to the total number of moles of the typical metal group 1 (Sn/(typical metal group 1)) is preferably from 0.20 to 0.40, more preferably from 0.25 to 0.40, and still more preferably from 0.25 to 0.35. When the (Sn/(typical metal group 1)) ratio is equal to or greater than the lower limit value of the above range, the catalyst life improves. When the (Sn/(typical metal group 1)) ratio is equal to or less than the upper limit value of the above range, the catalytic activity improves.

The average particle size of the six element alloy serving as the active component of the dehydrogenation catalyst 2-1 is preferably from 0.1 to 4 nm, more preferably from 0.1 to 3 nm, and still more preferably from 0.1 to 2.2 nm. When the average particle size of the six element alloy is equal to or greater than the lower limit value of the above range, the catalyst life improves. When the average particle size of the six element alloy is equal to or less than the upper limit value of the above range, the catalytic activity improves. The average particle size of the six element alloy can be measured by TEM and STEM. More specifically, it can be measured by the same method as the method for measuring the average particle size of the aforementioned complex, which is the active component of the aforementioned dehydrogenation catalyst 1.

As described above, the Pt₃ site in which three platinum atoms are in contact with each other and the Pt₁ site in which one platinum atom is surrounded by three gallium atoms are present on the surface of the platinum-gallium alloy of the dehydrogenation catalyst 1, and not only the dehydrogenation reaction of propane but also the side reaction of producing coke occurs at the Pt₃ site. In the dehydrogenation catalyst 1, the side reaction is suppressed due to the covering of the Pt₃ site with the lead atoms. In this case, it becomes difficult to effectively utilize Pt derived from the covered Pt₃ site.

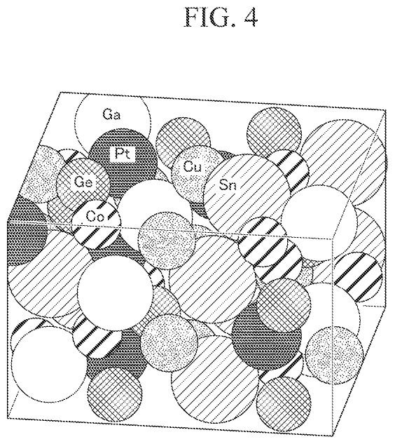

FIG. 4 shows a crystal structure (unit cell) of the six element alloy. As shown in FIG. 4, the six element alloy in the dehydrogenation catalyst 2-1 has a unique crystal structure. By forming a six element alloy, it is possible to obtain an active component having a structure in which the Pt₃ site where three platinum atoms are in contact with each other is substantially absent, and only the Pt₁ site where only one platinum atom is exposed is substantially present.

In the case of having the crystal structure shown in FIG. 4, a peak attributed to (PtGe) is confirmed at a position of 20=42 to 470 in an X-ray diffraction pattern.

The X-ray diffraction pattern of the dehydrogenation catalyst can be obtained by powder X-ray diffraction measurement using CuKα as a radiation source. For example, for a powder catalyst for dehydrogenation, an X-ray diffraction pattern can be obtained by using an X-ray diffractometer (for example, MiniFlex II/AP manufactured by Rigaku Corporation).

### Embodiment 2-2

In the case of the second dehydrogenation catalyst, the active component of the dehydrogenation catalyst of the embodiment 2-2 (hereinafter, also referred to as “dehydrogenation catalyst 2-2”) contains a platinum element, and a gallium element, a cobalt element, a copper element and an iron element serving as the elements M1. The element M1 may contain an element other than the above-mentioned elements, but the element M1 is preferably composed only of the above-mentioned elements. Further, an element M2 may be contained.

In the active component of the dehydrogenation catalyst 2-2, the platinum element, the gallium element, the cobalt element, the copper element and the iron element form an alloy. That is, the aforementioned active component preferably contains a platinum-gallium-cobalt-copper-iron alloy (hereinafter, also referred to as “five element alloy”).

The content ratio of the aforementioned active component with respect to the total mass of the dehydrogenation catalyst 2-2 is preferably from 0.3 to 28% by mass, more preferably from 0.3 to 18% by mass, and still more preferably from 0.3 to 11.5% by mass. When the content ratio of the active component is equal to or greater than the lower limit value of the above range, the catalyst life improves. When the content ratio of the active component is equal to or less than the upper limit value of the above range, the catalyst life improves.

The content ratio of the platinum element with respect to the total mass of the dehydrogenation catalyst 2-2 is preferably from 0.1 to 5% by mass, more preferably from 0.1 to 3% by mass, and still more preferably from 0.1 to 1% by mass. When the content ratio of the platinum element is equal to or greater than the lower limit value of the above range, the catalyst life improves. When the content ratio of the platinum element is equal to or less than the upper limit value of the above range, the catalyst life improves.

The content ratio of the gallium element with respect to the total mass of the dehydrogenation catalyst 2-2 is preferably from 0.05 to 5% by mass, more preferably from 0.05 to 3% by mass, and still more preferably from 0.05 to 2% by mass. When the content ratio of the gallium element is equal to or greater than the lower limit value of the above range, the catalyst life improves. When the content ratio of the gallium element is equal to or less than the upper limit value of the above range, the catalytic activity improves.

The content ratio of the cobalt element with respect to the total mass of the dehydrogenation catalyst 2-2 is preferably from 0.03 to 3% by mass, more preferably from 0.03 to 2% by mass, and still more preferably from 0.03 to 1.5% by mass. When the content ratio of the cobalt element is equal to or greater than the lower limit value of the above range, the catalyst life improves. When the content ratio of the cobalt element is equal to or less than the upper limit value of the above range, the catalytic activity improves.

The content ratio of the copper element with respect to the total mass of the dehydrogenation catalyst 2-2 is preferably from 0.03 to 4% by mass, more preferably from 0.03 to 3% by mass, and still more preferably from 0.03 to 2% by mass. When the content ratio of the copper element is equal to or greater than the lower limit value of the above range, the catalyst life improves. When the content ratio of the copper element is equal to or less than the upper limit value of the above range, the catalytic activity improves.

The content ratio of the iron element with respect to the total mass of the dehydrogenation catalyst 2-2 is preferably from 0.03 to 3% by mass, more preferably from 0.03 to 2% by mass, and still more preferably from 0.03 to 1.5% by mass. When the content ratio of the iron element is equal to or greater than the lower limit value of the above range, the catalyst life improves. When the content ratio of the iron element is equal to or less than the upper limit value of the above range, the catalytic activity improves.

When the inventors of the present invention analyzed the crystal structure of the five element alloy, it was found to have a random face-centered cubic lattice (fcc) structure. The random fcc structure is a structure in which the above five elements are randomly present at each site in the fcc structure. Hereinafter, platinum, cobalt, copper and iron are collectively referred to as “transition metal group 2”.

The ratio of the number of moles of gallium with respect to the total number of moles of the transition metal group 2 (Ga/(transition metal group 2)) is preferably from 0.3 to 0.4, more preferably from 0.35 to 0.37, and still more preferably from 0.35 to 0.355. When the (Ga/(transition metal group 2)) ratio is equal to or greater than the lower limit value of the above range, the catalyst life improves. When the (Ga/(transition metal group 2)) ratio is equal to or less than the upper limit value of the above range, the catalytic activity improves.

The ratio of the number of moles of the platinum element with respect to the total number of moles of the transition metal group 2 (Pt/(transition metal group 2)) is preferably from 0.05 to 0.15, more preferably from 0.1 to 0.15, and still more preferably from 0.11 to 0.12. When the (Pt/(transition metal group 2)) ratio is equal to or greater than the lower limit value of the above range, the catalytic activity improves. When the (Pt/(transition metal group 2)) ratio is equal to or less than the upper limit value of the above range, the catalyst life improves.

The ratio of the number of moles of the cobalt element with respect to the total number of moles of the transition metal group 2 (Co/(transition metal group 2)) is preferably from 0.15 to 0.2, more preferably from 0.16 to 0.19, and still more preferably from 0.17 to 0.18. When the (Co/(transition metal group 2)) ratio is equal to or greater than the lower limit value of the above range, the catalyst life improves. When the (Co/(transition metal group 2)) ratio is equal to or less than the upper limit value of the above range, the catalytic activity improves.

The ratio of the number of moles of the copper element with respect to the total number of moles of the transition metal group 2 (Cu/(transition metal group 2)) is preferably from 0.15 to 0.2, more preferably from 0.16 to 0.19, and still more preferably from 0.17 to 0.18. When the (Cu/(transition metal group 2)) ratio is equal to or greater than the lower limit value of the above range, the catalyst life improves. When the (Cu/(transition metal group 2)) ratio is equal to or less than the upper limit value of the above range, the catalytic activity improves.

The ratio of the number of moles of the iron element with respect to the total number of moles of the transition metal group 2 (Fe/(transition metal group 2)) is preferably from 0.15 to 0.2, more preferably from 0.16 to 0.19, and still more preferably from 0.17 to 0.18. When the (Fe/(transition metal group 2))ratio is equal to or greater than the lower limit value of the above range, the catalyst life improves. When the (Fe/(transition metal group 2)) ratio is equal to or less than the upper limit value of the above range, the catalytic activity improves.

An average particle size of the five element alloy serving as the active component of the dehydrogenation catalyst 2-2 is preferably from 1 to 7 nm, more preferably from 1 to 5 nm, and still more preferably from 1 to 3 nm. When the average particle size of the five element alloy is equal to or greater than the lower limit value of the above range, the catalyst life improves. When the average particle size of the five element alloy is equal to or less than the upper limit value of the above range, the catalytic activity improves. The average particle size of the five element alloy can be measured by TEM and STEM. More specifically, it can be measured by the same method as the method for measuring the average particle size of the aforementioned complex, which is the active component of the aforementioned dehydrogenation catalyst 1.

In the case of having a random fcc structure, a peak attributed to the (111) plane is confirmed at a position of 2θ=36 to 440 in an X-ray diffraction pattern.

### Third Embodiment

In the case of the first dehydrogenation catalyst, the dehydrogenation catalyst of the third embodiment (hereinafter, also referred to as “dehydrogenation catalyst 3”) contains a calcium element as the element M. In the case of the second dehydrogenation catalyst, the dehydrogenation catalyst 3 contains a calcium element as the element M2.

Hereinafter, particularly preferred embodiments of the dehydrogenation catalyst 3 will be described.

In the case of an embodiment, the active component of the dehydrogenation catalyst 3 contains a platinum element, a gallium element, and a calcium element serving as the element M in the case of the first dehydrogenation catalyst. As the element M, an element other than calcium may be contained. In the case of an embodiment, it is preferable to contain only the calcium element as the element M. In the case of the second dehydrogenation catalyst, the active component of the dehydrogenation catalyst 3 contains platinum, a gallium element as the element M1, and a calcium element as the element M2.

Hereinafter, the dehydrogenation catalyst 3 containing a platinum element, a gallium element and a calcium element is also referred to as “dehydrogenation catalyst 3-1”.

In the case of another embodiment, it is preferable that the active component of the dehydrogenation catalyst 3 further contains a lead element as the element M in the case of the first dehydrogenation catalyst. In the case of the second dehydrogenation catalyst, it is preferable that the active component of the dehydrogenation catalyst 3 further contains a lead element as the element M2.

Hereinafter, the dehydrogenation catalyst 3 containing a platinum element, a gallium element, a lead element and a calcium element is also referred to as “dehydrogenation catalyst 3-2”. The dehydrogenation catalyst 3-2 is an embodiment of the above-mentioned dehydrogenation catalyst 1 that further contains a calcium element.

In the case of yet another embodiment, it is preferable that the active component of the dehydrogenation catalyst 3 contains a gallium element, a cobalt element and a copper element as the element M1, and a calcium element as the element M2 in the case of the second dehydrogenation catalyst.

Hereinafter, the dehydrogenation catalyst 3 containing a platinum element, a gallium element, a cobalt element, a copper element and a calcium element is also referred to as “dehydrogenation catalyst 3-A”. The dehydrogenation catalyst 3-A is an embodiment of the above-mentioned dehydrogenation catalyst 2 that further contains a calcium element.

In the case of yet another embodiment, it is preferable that the active component of the dehydrogenation catalyst 3 contains a cobalt element, a copper element, a germanium element, a tin element and a calcium element as the elements M in the case of the first dehydrogenation catalyst. In the case of the second dehydrogenation catalyst, it is preferable that the active component of the dehydrogenation catalyst 3 contains a gallium element, a cobalt element, a copper element, a germanium element and a tin element as the elements M1, and a calcium element as the element M2.

Hereinafter, the dehydrogenation catalyst 3 containing a platinum element, a gallium element, a cobalt element, a copper element, a germanium element, a tin element and a calcium element is also referred to as “dehydrogenation catalyst 3-3”. The dehydrogenation catalyst 3-3 is an embodiment of the above-mentioned dehydrogenation catalyst 2-1 that further contains a calcium element.

In the case of yet another embodiment, it is preferable that the active component of the dehydrogenation catalyst 3 contains a gallium element, a cobalt element, a copper element and an iron element as the elements M1 and a calcium element as the element M2 in the second dehydrogenation catalyst.

Hereinafter, the dehydrogenation catalyst 3 containing a platinum element, a gallium element, a cobalt element, a copper element, an iron element and a calcium element is also referred to as “dehydrogenation catalyst 3-4”. The dehydrogenation catalyst 3-4 is an embodiment of the above-mentioned dehydrogenation catalyst 2-2 that further contains a calcium element.

In the case of yet another embodiment, it is preferable that the active component of the dehydrogenation catalyst 3 contains a copper element as the element M1 and a calcium element as the element M2 in the second dehydrogenation catalyst. It is preferable to contain only the copper element as the element M1. It is preferable to contain only the calcium element as the element M2.

Hereinafter, the dehydrogenation catalyst 3 containing a platinum element, a copper element and a calcium element is also referred to as “dehydrogenation catalyst 3-5”.

The content ratio of the aforementioned active component with respect to the total mass of the dehydrogenation catalyst 3-5 is preferably from 1.1 to 28% by mass, more preferably from 1.1 to 18% by mass, and still more preferably from 1.1 to 11.5% by mass. When the content ratio of the active component is equal to or greater than the lower limit value of the above range, the catalyst life improves. When the content ratio of the active component is equal to or less than the upper limit value of the above range, the catalyst life improves.

The content ratio of the platinum element with respect to the total mass of the dehydrogenation catalyst 3-5 is preferably from 0.1 to 5% by mass, more preferably from 0.1 to 3% by mass, and still more preferably from 0.1 to 1% by mass. When the content ratio of the platinum element is equal to or greater than the lower limit value of the above range, the catalyst life improves. When the content ratio of the platinum element is equal to or less than the upper limit value of the above range, the catalyst life improves.

The content ratio of the copper element with respect to the total mass of the dehydrogenation catalyst 3-5 is preferably from 1 to 20% by mass, more preferably from 1 to 15% by mass, and still more preferably from 1 to 10% by mass. When the content ratio of the copper element is equal to or greater than the lower limit value of the above range, the catalyst life improves. When the content ratio of the copper element is equal to or less than the upper limit value of the above range, the catalytic activity improves.

The molar ratio of the copper element with respect to the platinum element (Cu/Pt) in the dehydrogenation catalyst 3-5 is preferably from 15 to 50, more preferably from 20 to 50, and still more preferably from 25 to 50. When the Cu/Pt ratio is equal to or greater than the lower limit value of the above range, the propylene selectivity improves. When the Cu/Pt ratio is equal to or less than the upper limit value of the above range, the propylene selectivity improves.

The form of the calcium element in the dehydrogenation catalyst 3 is not particularly limited, but is preferably an oxide.

In the case of containing only a calcium element as the element M (that is, when the dehydrogenation catalyst 3-1 is used), the platinum element and the gallium element preferably form an alloy in the active component of the dehydrogenation catalyst 3-1. Preferred embodiments of the platinum-gallium alloy are as described above. That is, the active component of the dehydrogenation catalyst 3-1 is preferably a complex of a platinum-gallium alloy and calcium oxide.

In this case, the molar ratio of the calcium element with respect to the platinum element (Ca/Pt) in the dehydrogenation catalyst 3-1 is preferably from 3 to 7, more preferably from 3 to 5, and still more preferably from 4 to 5. When the Ca/Pt ratio is equal to or greater than the lower limit value of the above range, the catalyst life improves. When the Ca/Pt ratio is equal to or less than the upper limit value of the above range, the catalytic activity improves.

In the case of containing a calcium element and a lead element as the elements M (that is, when the dehydrogenation catalyst 3-2 is used), the active component of the dehydrogenation catalyst 3-2 preferably contains a complex containing a complex in which the lead element (lead atom) contained in the active component of the dehydrogenation catalyst 1 described above is present on the surface of the platinum-gallium alloy and calcium oxide.

In this case, the molar ratio of the calcium element with respect to the platinum element (Ca/Pt) in the dehydrogenation catalyst 3-2 is preferably from 3 to 7, more preferably from 3 to 5, and still more preferably from 4 to 5. When the Ca/Pt ratio is equal to or greater than the lower limit value of the above range, the catalyst life improves. When the Ca/Pt ratio is equal to or less than the upper limit value of the above range, the catalytic activity improves.

In the case of containing a calcium element, a cobalt element, a copper element, a germanium element and a tin element as the elements M (that is, when the dehydrogenation catalyst 3-3 is used), the active component of the dehydrogenation catalyst 3-3 is preferably a complex containing a six element alloy contained in the active component of the above-mentioned dehydrogenation catalyst 2-1 and calcium oxide.

In this case, the molar ratio of the calcium element with respect to the platinum element (Ca/Pt) in the dehydrogenation catalyst 3-3 is preferably from 9 to 20, more preferably from 11 to 18, and still more preferably from 12 to 17. When the Ca/Pt ratio is equal to or greater than the lower limit value of the above range, the catalyst life improves. When the Ca/Pt ratio is equal to or less than the upper limit value of the above range, the catalytic activity improves.

In the case of the dehydrogenation catalyst 3-4, the active component of the dehydrogenation catalyst 3-4 is preferably a complex containing a five element alloy contained in the active component of the above-mentioned dehydrogenation catalyst 2-2 and calcium oxide.

In this case, the molar ratio of the calcium element with respect to the platinum element (Ca/Pt) in the dehydrogenation catalyst 3-4 is preferably from 10 to 20, more preferably from 12 to 18, and still more preferably from 14 to 16. When the Ca/Pt ratio is equal to or greater than the lower limit value of the above range, the catalyst life improves. When the Ca/Pt ratio is equal to or less than the upper limit value of the above range, the catalytic activity improves.

In the case of the dehydrogenation catalyst 3-5, the active component of the dehydrogenation catalyst 3-5 is preferably a complex of a platinum-copper alloy and calcium oxide.

In this case, the molar ratio of the calcium element with respect to the platinum element (Ca/Pt) in the dehydrogenation catalyst 3-5 is preferably from 10 to 20, more preferably from 12 to 18, and still more preferably from 14 to 16. When the Ca/Pt ratio is equal to or greater than the lower limit value of the above range, the catalyst life improves. When the Ca/Pt ratio is equal to or less than the upper limit value of the above range, the catalytic activity improves.

In the case of yet another embodiment, it is preferable that the active component of the dehydrogenation catalyst 3 further contains a copper element and a cobalt element as the elements M in the case of the first dehydrogenation catalyst. In the case of the second dehydrogenation catalyst, the active component of the dehydrogenation catalyst 3 preferably contains a gallium element, a copper element and a cobalt element as the elements M1.

In the case of the dehydrogenation catalyst 3-A, the active component of the dehydrogenation catalyst 3-A is preferably a complex containing the alloy contained in the active component of the above-mentioned dehydrogenation catalyst 2 and calcium oxide.

In this case, as for the preferred range of the molar ratio of the calcium element with respect to the platinum element (Ca/Pt) in the dehydrogenation catalyst 3-A, the ranges described for the above-mentioned dehydrogenation catalysts 3-3 and 3-4 can be applied.

The role of the calcium element in the dehydrogenation catalyst 3 is presumed to be an electronic effect that improves the selectivity and durability of the Pt₁ site described above. It should be noted that although the inventors of the present application have also conducted intensive studies on alkaline earth metals other than the calcium element, alkali metals and the like which have properties similar to those of the calcium element, it was found that such an effect can be obtained only by the calcium element.

<<Method for producing dehydrogenation catalys>>

A method for producing a dehydrogenation catalyst of the present embodiment includes: an impregnation step of impregnating a silica carrier with an impregnating solution containing a raw material compound of the aforementioned active component to obtain an impregnated body; and a reduction calcining step of subjecting the aforementioned impregnated body to reduction calcining in a reducing gas atmosphere and/or an oxidation calcining step of subjecting the aforementioned impregnated body to oxidation calcining in an oxidizing gas atmosphere.

### <Impregnation Step>

A raw material compound containing a platinum element, a raw material compound containing a gallium element and a raw material compound containing an element M that are used in the impregnation step of producing a first dehydrogenation catalyst, and a raw material compound containing a platinum element, a raw material compound containing an element M1 and a raw material compound containing an element M2 that are used in the impregnation step of producing a second dehydrogenation catalyst (hereinafter, all the raw material compounds are collectively referred to as “raw material compound of the active component”) are not particularly limited, and examples thereof include inorganic salts such as chlorides, sulfides, nitrates and carbonates; organic salts such as oxalates, acetylacetonate salts, dimethylglyoxime salts and ethylenediamine acetates; chelate compounds; carbonyl compounds; cyclopentadienyl compounds; ammine complexes; alkoxide compounds; and alkyl compounds.

Examples of the impregnation method include: a method of evaporation to dryness for supporting an active component by immersing a silica carrier in an impregnating solution that is in excess with respect to the total pore volume of the silica carrier and then drying all the solvent in a drying step described later; an equilibrium adsorption method for obtaining a catalyst carrying an active component by immersing a silica carrier in an impregnating solution that is in excess with respect to the total pore volume of the silica carrier, subjecting the resultant to a solid-liquid separation process such as filtration, and then drying the solvent; and a pore filling method for supporting an active component by impregnating a silica carrier with an impregnating solution in an amount substantially equal to the total pore volume of the silica carrier and drying all the solvent in a drying step described later. It should be noted that as a method of impregnating a silica carrier with two or more types of raw material compounds of active components, a batch impregnation method of simultaneously impregnating each of these components or a sequential impregnation method of individually impregnating each of these components may be employed.

The impregnating solution can be prepared by dissolving the raw material compound of the active component in a solvent. The solvent is not particularly limited as long as it can dissolve the raw material compound of the active component and is volatilized and removed by the drying step described later, and examples thereof include water, ethanol and acetone.

The solvent in the impregnating solution can be dried by a method known in the art, and the drying temperature, drying time and drying atmosphere can be appropriately adjusted depending on the solvent to be removed.

Examples of the reducing gas in the reduction calcining step include hydrogen, carbon monoxide and the like, and a gas diluted with an inert gas may be used. The reduction calcining temperature is preferably from 500 to 800° C., more preferably from 500 to 700° C., and still more preferably from 600 to 700° C.

The reduction calcining time may be from 0.2 to 3 hours, 0.5 to 2 hours, or 0.5 to 1 hour.

Examples of the oxidizing gas in the oxidation calcining step include oxygen and air, and a gas diluted with an inert gas may be used. The oxidation calcining temperature is preferably from 200 to 600° C., more preferably from 200 to 500° C., and still more preferably from 200 to 400° C.

The oxidation time may be from 0.5 to 3 hours, 0.5 to 2 hours, or 0.5 to 1 hour.

In the first dehydrogenation catalyst, in the case of producing the dehydrogenation catalyst 1, a raw material compound containing a lead element is used as the raw material compound containing the element M. In the case of producing the dehydrogenation catalyst 2-1, as the raw material compound containing the element M, a raw material compound containing a cobalt element, a raw material compound containing a copper element, a raw material compound containing a germanium element, and a raw material compound containing a tin element am used. In the case of producing the dehydrogenation catalyst 3, a raw material compound containing a calcium element is used as the raw material compound containing the element M.

In the second dehydrogenation catalyst, in the case of producing the dehydrogenation catalyst 1, a raw material compound containing a gallium element is used as the raw material compound containing the element M1, and a raw material compound containing a lead element is used as the raw material compound containing the element M2. In the case of producing the dehydrogenation catalyst 2, a raw material compound containing a gallium element, a raw material compound containing a cobalt element and a raw material compound containing a tin element are used as the raw material compounds containing the element M1. In the case of producing the dehydrogenation catalyst 2-1, a raw material compound containing a gallium element, a raw material compound containing a cobalt element, a raw material compound containing a copper element, a raw material compound containing a germanium element and a raw material compound containing a tin element are used as the raw material compounds containing the element M1. In the case of producing the dehydrogenation catalyst 2-2, a raw material compound containing a gallium element, a raw material compound containing a cobalt element, a raw material compound containing a copper element and a raw material compound containing an iron element are used as the raw material compounds containing the element M1. In the case of producing the dehydrogenation catalyst 3, a raw material compound containing a calcium element is used as the raw material compound containing the element M2.

### <<Method for Producing Propylene>>

A method for producing propylene of the present embodiment is a method in which the dehydrogenation catalyst of the present invention and a source gas containing propane are subjected to a contact treatment to produce propylene by a dehydrogenation reaction of propane.

The method for producing propylene can be carried out, for example, by filling a reactor with the above-mentioned dehydrogenation catalyst and circulating the source gas containing propane. A reaction system is not particularly limited as long as the effects of the present invention can be obtained, and examples thereof include a fixed bed system, a fluidized bed system, and a moving bed system, and a fixed bed system is preferred.

The method for producing propylene may be a one-step propylene production method carried out by filling a single reaction device with the above-mentioned dehydrogenation catalyst, or a multi-step continuous propylene production method carried out by filling a plurality of reaction devices.

The content ratio of propane with respect to 100% by volume of the source gas is preferably from 20 to 100% by volume, and more preferably from 50 to 100% by volume. Examples of the gas other than propane in the source gas include inert gases such as helium and nitrogen.

The source gas may contain hydrogen. The inclusion of hydrogen in the source gas suppresses the production of coke. The content ratio of hydrogen with respect to 100% by volume of the source gas is preferably from 10 to 40% by volume, and more preferably from 10 to 20% by volume. When the content ratio of hydrogen is equal to or greater than the lower limit value of the above range, the catalyst life improves. When the content ratio of hydrogen is equal to or less than the upper limit value of the above range, the catalytic activity improves.

The reaction temperature is preferably from 550 to 650° C., and more preferably from 580 to 620° C. When the reaction temperature is equal to or higher than the lower limit value of the above range, the equilibrium conversion rate increases. When the reaction temperature is equal to or lower than the upper limit value of the above range, the sintering of the active component is suppressed and the decrease in activity is suppressed.

The reaction pressure is preferably from 0.1 to 0.3 MPa, more preferably from 0.1 to 0.25 MPa, and still more preferably from 0.1 to 0.2 MPa.

The weight hourly space velocity (WHSV) of propane in the source gas with respect to the dehydrogenation catalyst is more preferably from 2 to 4 hr⁻¹, and still more preferably from 2 to 3 hr⁻¹. When the WHSV value is equal to or greater than the lower limit value of the above range, the productivity improves.

Examples of the propane in the source gas used in the method for producing propylene of the present embodiment include propane derived from shale gas, propane derived from naphtha, and propane derived from biomass.

By using the dehydrogenation catalyst of the present invention, propylene can be produced for a longer period of time.

### EXAMPLES

Hereinafter, the present invention will be described in more detail with reference to Examples and Comparative Examples, but the present invention is not limited to the following Examples.

### <Characterization of Dehydrogenation Catalyst>

For the characterization of dehydrogenation catalysts, scanning transmission electron microscopy observation and CO adsorption analysis were performed.

### (Scanning Transmission Electron Microscopy Observation)

The particle size of the active component of the dehydrogenation catalyst in each example was measured by a scanning transmission electron microscope (FEI Titan G-2) equipped with an energy dispersive X-ray (EDX) analyzer at an acceleration voltage of 300 kV. The dehydrogenation catalyst of each example was subjected to an ultrasonic treatment with ethanol, and then dispersed and observed on a Mo grid supported by a carbon film. The longest diameters of 100 or more particles (active components) randomly selected from 10 images were observed, and an average of these values was taken as the average particle size. The average particle sizes of the dehydrogenation catalysts observed with a scanning transmission electron microscope are shown in Tables 2, 3 and 5.

### (CO Adsorption)

The dispersity of platinum in the dehydrogenation catalyst was measured by measuring the CO adsorption on the dehydrogenation catalyst in each example. The dispersity refers to a ratio of platinum exposed on the surface with respect to the total amount of platinum contained in the dehydrogenation catalyst. After a pretreatment at 600° C. for 30 minutes under a circulation of a mixed gas composed of 5% by volume of hydrogen and 95% by volume of argon at 40 NmL/min, 50 to 100 mg of the dehydrogenation catalyst was cooled with liquid nitrogen while being purged with helium. Next, a mixed gas composed of 10% by volume of carbon monoxide and 90% by volume of helium was introduced by a pulse method, carbon monoxide that was not adsorbed on the catalyst was quantified by a TCD detector, and the mixed gas was introduced until no carbon monoxide was adsorbed on the catalyst. The dispersity of platinum was calculated from the amount of carbon monoxide adsorbed on the dehydrogenation catalyst on the assumption that one molecule of carbon monoxide was adsorbed on one atom of platinum. Tables 2 and 3 show the results of Pt dispersity for the dehydrogenation catalysts for which the CO adsorption measurement was performed.

### <Dehydrogenation Reaction of Propane>

The dehydrogenation catalyst of each example was diluted with quartz sand as needed, and filled in a quartz cylindrical fixed bed reaction tube having a diameter of 6 mm and a length of 30 cm to form a catalyst layer. Subsequently, hydrogen was circulated through the catalyst layer to perform a pretreatment. Then, a source gas containing propane was circulated through the catalyst layer to carry out a dehydrogenation reaction of propane. Detailed reaction conditions are shown in Table 1.

TABLE 1

| | | Reaction | | Reaction | | Reaction | | Reaction | |
|---|---|---|---|---|---|---|---|---|---|
| | | condition 1 | | condition 2 | | condition 3 | | condition 4 | |
| Filling amount | Catalyst | 15 | mg | 30 | mg | 10 | mg | 100 | mg |
| | Quartz sand | 1,485 | mg | 0 | mg | 0 | mg | 0 | mg |
| Pretreatment | H₂ | 10N | mL/min | 10N | mL/min | 10N | mL/min | 10N | mL/min |
| | Temperature | 600° | C. | 600° | C. | 600° | C. | 600° | C. |
| | Time | 0.5 | h | 0.5 | h | 0.5 | h | 0.5 | h |
| Reaction | Reactant gas composition | 3.9:5:40 | | 2.5:0:5.0 | | 2.5:0:5.0 | | 2.5:0:5.0 | |
| | (C₃H₈:H₂:He) | | | | | | | | |
| | Reactant gas flow rate | 48.9N | mL/min | 7.5N | mL/min | 7.5N | mL/min | 7.5N | mL/min |
| | WHSV of C₃H₈ | 30.7 | h⁻¹ | 9.8 | h⁻¹ | 29.5 | h⁻¹ | 3.0 | h⁻¹ |
| | Reaction temperature | 600° | C. | 600° | C. | 600° | C. | 600° | C. |

| | | Reaction | | Reaction | | Reaction | | Reaction | |
|---|---|---|---|---|---|---|---|---|---|
| | | condition 5 | | condition 6 | | condition 7 | | condition 8 | |
| Filling amount | Catalyst | 20 | mg | 150 | mg | 60 | mg | 50 | mg |
| | Quartz sand | 0 | mg | 0 | mg | 0 | mg | 0 | mg |
| Pretreatment | H₂ | 10N | mL/min | 10N | mL/min | 10N | mL/min | 10N | mL/min |
| | Temperature | 600° | C. | 600° | C. | 600° | C. | 600° | C. |
| | Time | 0.5 | h | 0.5 | h | 0.5 | h | 0.5 | h |

| | | | | | |
|---|---|---|---|---|---|
| Reaction | Reactant gas composition | 2.5:0:5.0 | 2.5:1.3:3.7 | 2.5:0: 5.0 | 2.5:0:5.0 |
| | (C₃H₈:H₂:He) | | | | |

| | | | | | | | | |
|---|---|---|---|---|---|---|---|---|
| Reactant gas flow rate | 7.5N | mL/min | 7.5N | mL/min | 7.5N | mL/min | 7.5N | mL/min |
| WHSV of C₃H₈ | 14.8 | h⁻¹ | 2.0 | h⁻¹ | 5.9 | h⁻¹ | 4.9 | h⁻¹ |
| Reaction temperature | 600° | C. | 600° | C. | 600° | C. | 600° | C. |

A gas discharged from a reactor was analyzed by an online thermal conductivity detection gas chromatograph (product name “GC-8A”, manufactured by Shimadzu Corporation). Propylene, propane, ethylene, ethane, and methane were detected in the reactor outlet gas.

The conversion rate of propane was calculated by the following equation (1).

$$[ Equation ( 1 ) ]$$

$${C}_{3} {H}_{8} conversion ( \% ) = \frac{{[ {C}_{3} {H}_{8} ]}_{inlet} - {[ {C}_{3} {H}_{8} ]}_{out let}}{{[ {C}_{3} {H}_{8} ]}_{inlet}} ⨯ 100 \qquad ( 1 )$$

In the above equation (1), [C₃H₈]ᵢₙₗₑₜ indicates a flow rate (mol/min) of propane supplied to the reactor, and [C₃H₈]ₒᵤₜₗₑₜ indicates a flow rate (mol/min) of propane discharged from the reactor.

The selectivity of propylene was calculated by the following equation (2).

$$[ Equation ( 2 ) ]$$

$${C}_{3} {H}_{6} selectivity ( \% ) = \frac{[ {C}_{3} {H}_{6} ]}{[ {C}_{3} {H}_{6} ] + \frac{2}{3} [ {C}_{2} {H}_{6} ] + \frac{2}{3} [ {C}_{2} {H}_{4} ] + \frac{1}{3} [ {CH}_{4} ]} ⨯ 100 \qquad ( 2 )$$

In the above equation (2), [C₃H₆] indicates a flow rate (mol/min) of propylene discharged from the reactor, [C₂H₆] indicates a flow rate (mol/min) of ethane discharged from the reactor, [C₂H₄] indicates a flow rate (mol/min) of ethylene discharged from the reactor, and [CH₄] indicates a flow rate (mol/min) of methane discharged from the reactor.

The yield of propylene was calculated by a formula: [flow rate (mol/min) of propylene discharged from the reactor]/[flow rate (mol/min) of propane supplied to the reactor]×100.

The average catalyst life of the dehydrogenation catalyst was calculated by a primary deactivation model. More specifically, the average catalyst life of the dehydrogenation catalyst was calculated by the following equations (3) and (4).

$$[ Equation ( 3 ) ]$$

$${k}_{d} = \frac{ln ( \frac{1 - c onv {.}_{e n d}}{conv {.}_{e n d}} ) - ln ( \frac{1 - conv {.}_{start}}{c onv {.}_{s t a r t}} )}{t} \qquad ( 3 )$$

In the above equation (3), k_{d} indicates a deactivation rate constant (h⁻¹), t indicates a reaction time (h), convₛₜₐᵣₜ indicates a conversion rate (%) of propane at the start of the reaction, and conv_{end} indicates a conversion rate (%) of propane at a reaction time t(h).

$$[ Equation ( 4 ) ]$$

$$τ = \frac{1}{{k}_{d}} \qquad ( 4 )$$

In the above equation (4), τ indicates an average catalyst life (h).

<Measurement of Carbon Content of Dehydrogenation Catalyst after Reaction>

The amount of carbon of the dehydrogenation catalyst after the reaction in each example was measured by BELCAT II manufactured by MicrotracBEL Corp. After a pretreatment at 300° C. for 30 minutes under a circulation of helium at 20 NmL/min. 10 mg of the dehydrogenation catalyst (excluding quartz sand) after the reaction for 20 hours was cooled to room temperature. Next, the temperature was increased from 100 to 800° C. at a rate of 2° C./min while circulating a mixed gas composed of 2% by volume of oxygen and 98% by volume of helium at 50 NmL/min. The amount of carbon dioxide in the outlet gas was quantified by an online mass spectrometer. It should be noted that a relative ratio of the amount of carbon is shown when the unit in the table is denoted as au., and when the unit is denoted as g_{coke}/g_{cat}, it means an absolute value of the amount of carbon per 1 g of the catalyst.

### Example 1

Silica (product name “CARiACT G-6”, manufactured by Fuji Silysia Chemical Ltd., specific surface area: 673 m²/g) was impregnated with an impregnating solution prepared by dissolving Pt(NH₃)₂(NO₃)₂ as a Pt source, Ga(NO₃)₃.nH₂O (n=7 to 9) as a Ga source and Pb(NO₃)₂ as a Pb source in ion-exchanged water so as to achieve the composition of the catalyst 1 shown in Table 2 by a pore filling method. The obtained impregnated body was stored overnight at room temperature in a sealed round bottom flask, and subsequently frozen with liquid nitrogen and freeze dried in vacuum at −5° C. The obtained powder was further dried overnight in an oven at 90° C., followed by calcining at 400° C. for 1 hour in an air atmosphere, and then subjected to reduction calcining at 700° C. for 1 hour while circulating hydrogen (0.1 MPa, 50 NmL/min) to obtain a catalyst 1 in which Pt, Ga and Pb were supported on silica.

### Comparative Example 1

A catalyst 2 in which Pt was supported on silica was obtained in the same manner as in Example 1 except that Ga(NO₃)₃.nH₂O (n=7 to 9) and Pb(NO₃)₂ were not added, and the composition of the impregnating solution was changed so as to achieve the composition of the catalyst 2 shown in Table 2.

### Comparative Example 2

A catalyst 3 in which Pt and Ga were supported on silica was obtained in the same manner as in Example 1 except that Pb(NO₃)₂ was not added, and the composition of the impregnating solution was changed so as to achieve the composition of the catalyst 3 shown in Table 2.

### Comparative Example 3

A catalyst 4 in which Pt and Ga were supported on silica was obtained in the same manner as in Example 1 except that Pb(NO₃)₂ was not added, and the composition of the impregnating solution was changed so as to achieve the composition of the catalyst 4 shown in Table 2.

### Comparative Example 4

A catalyst 5 in which Pt and Sn were supported on silica was obtained in the same manner as in Example 1 except that Ga(NO₃)₃.nH₂O (n=7 to 9) and Pb(NO₃)₂ were not added, SnCl₂ was added as a Sn source, and the composition of the impregnating solution was changed so as to achieve the composition of the catalyst 5 shown in Table 2.

### Comparative Example 5

A catalyst 6 in which Pt and Sn were supported on silica was obtained in the same manner as in Example 1 except that Ga(NO₃)₃.nH₂O (n=7 to 9) and Pb(NO₃)₂ were not added, SnCl₂ was added as a Sn source, and the composition of the impregnating solution was changed so as to achieve the composition of the catalyst 6 shown in Table 2.

### Comparative Example 61

A catalyst 7 in which Pt and In were supported on silica was obtained in the same manner as in Example 1 except that Ga(NO₃)₃.nH₂O (n=7 to 9) and Pb(NO₃)₂ were not added, In(NO₃)₃.nH₂O (n=8.8: measured by ICP) was added as an In source, and the composition of the impregnating solution was changed so as to achieve the composition of the catalyst 7 shown in Table 2.

### Comparative Example 71

A catalyst 8 in which Pt and Pb were supported on silica was obtained in the same manner as in Example 1 except that Ga(NO₃)₃.nH₂O (n=7 to 9) was not added, and the composition of the impregnating solution was changed so as to achieve the composition of the catalyst 8 shown in Table 2.

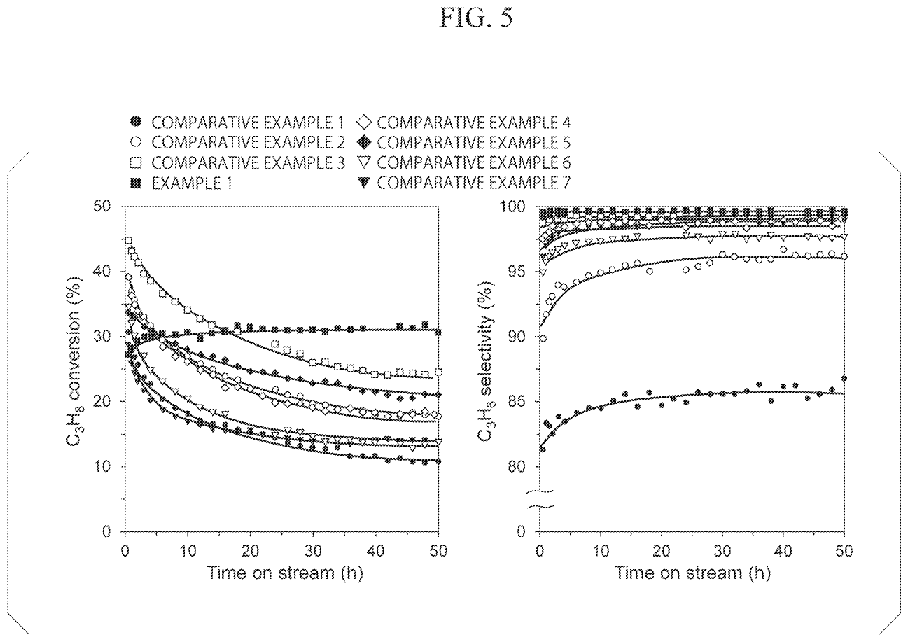

The dehydrogenation reactions of propane were carried out using the catalysts of Example 1 and Comparative Examples 1 to 7. It should be noted that the reaction condition 1 shown in Table 1 was employed as a reaction condition. The changes in the conversion rate of propane over time and the changes in the selectivity of propylene over time are shown in FIG. 5. The decrease in the conversion rate of propane over time was extremely suppressed in the catalyst of Example 1 containing Pt. Ga and Pb, as compared with the catalysts of Comparative Examples 1 to 7. It also showed a high propylene selectivity. Furthermore, as shown in Table 1, it was found that the carbon content of the catalyst after the reaction was extremely low.

### Example 2

A catalyst 9 in which Pt, Ga and Pb were supported on silica was obtained in the same manner as in Example 1 except that the composition of the impregnating solution was changed so as to achieve the composition of the catalyst 9 shown in Table 2.

### Example 3

A catalyst 10 in which Pt, Ga and Pb were supported on silica was obtained in the same manner as in Example 1 except that the composition of the impregnating solution was changed so as to achieve the composition of the catalyst 10 shown in Table 2.

### Example 4

A catalyst 11 in which Pt, Ga and Pb were supported on silica was obtained in the same manner as in Example 1 except that the composition of the impregnating solution was changed so as to achieve the composition of the catalyst 11 shown in Table 2.

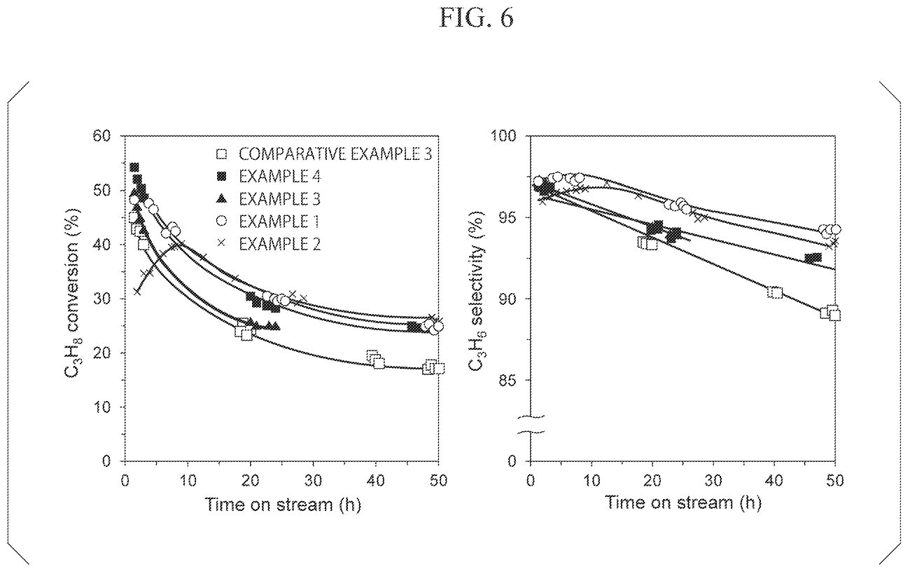

The dehydrogenation reactions of propane were carried out using the catalysts of Examples 1 to 4 and Comparative Example 3. It should be noted that the reaction condition 1 shown in Table 1 was employed as a reaction condition. However, the reaction temperature was set to 650° C. The changes in the conversion rate of propane over time and the changes in the selectivity of propylene over time are shown in FIG. 6. The decrease in the conversion rate of propane over time was suppressed in the catalysts of Examples 1 to 4 containing Pt Ga and Pb, as compared with the catalyst of Comparative Example 3. They also showed a high propylene selectivity.

TABLE 2

| | | | | | | | | | | Carbon | |
|---|---|---|---|---|---|---|---|---|---|---|---|
| | | | | | | | | | | content of | |
| | | | | | | | | | Average | catalyst | |
| | | | Amount of | | | | | | particle | after | Pt |
| | | | supported Pt | Molar ratio | | | | | size | reaction | dispersity |
| | Catalyst | Carrier | mass % | Pt | Ga | Pb | Sn | In | nm | a u | % |
| Ex. 1 | Catalyst 1 | SiO₂ | 3 | 1 | 1 | 0.5 | 0 | 0 | 2.8 | 9.6 | 5.9 |
| Comp. Ex. 1 | Catalyst 2 | SiO₂ | 3 | — | — | — | — | — | — | 100.0 | 37.1 |
| Comp. Ex. 2 | Catalyst 3 | SiO₂ | 3 | 1 | 0.33 | 0 | 0 | 0 | — | — | 24.9 |
| Comp. Ex. 3 | Catalyst 4 | SiO₂ | 3 | 1 | 1 | 0 | 0 | 0 | 2.9 | 81.7 | 9.9 |
| Comp. Ex. 4 | Catalyst 5 | SiO₂ | 3 | 1 | 0 | 0 | 0.5 | 0 | — | — | 24.2 |
| Comp. Ex. 5 | Catalyst 6 | SiO₂ | 3 | 1 | 0 | 0 | 1 | 0 | — | 54.3 | 8.0 |
| Comp. Ex. 6 | Catalyst 7 | SiO₂ | 3 | 1 | 0 | 0 | 0 | 0.33 | — | — | 20.2 |
| Comp. Ex. 7 | Catalyst 8 | SiO₂ | 3 | 1 | 0 | 0.5 | 0 | 0 | — | — | 17.5 |
| Ex. 2 | Catalyst 9 | SiO₂ | 3 | 1 | 1 | 0.67 | 0 | 0 | — | — | 2.5 |
| Ex. 3 | Catalyst 10 | SiO₂ | 3 | 1 | 1 | 0.40 | 0 | 0 | — | — | 4.6 |
| Ex. 4 | Catalyst 11 | SiO₂ | 3 | 1 | 1 | 0.20 | 0 | 0 | — | — | 8.5 |

### Example 5

Silica (product name “CARiACT G-6”, manufactured by Fuji Silysia Chemical Ltd., specific surface area: 673 m²/g) was impregnated with an impregnating solution prepared by dissolving Pt(NH₃)₂(NO₃)₂ as a Pt source, Ga(NO₃)₃.nH₂O (n=7 to 9) as a Ga source, Pb(NO₃)₂ as a Pb source and Ca(NO₃)₂.4H₂O as a Ca source in ion-exchanged water so as to achieve the composition of the catalyst 12 shown in Table 3 by a pore filling method. The obtained impregnated body was stored overnight at room temperature in a sealed round bottom flask, and subsequently frozen with liquid nitrogen and freeze dried in vacuum at −5° C. The obtained powder was further dried overnight in an oven at 90° C., followed by calcining at 600° C. for 1 hour in an air atmosphere, and then subjected to reduction calcining at 700° C. for 1 hour while circulating hydrogen (0.1 MPa, 50 NmL/min) to obtain a catalyst 12 in which Pt, Ga, Pb and Ca were supported on silica.

### Example 6

A catalyst 13 in which Pt, Ga and Ca were supported on silica was obtained in the same manner as in Example 5 except that Pb(NO₃)₂ was not added, and the composition of the impregnating solution was changed so as to achieve the composition of the catalyst 13 shown in Table 3.

### Example 7

A catalyst 14 in which Pt, Ga and Pb were supported on silica was obtained in the same manner as in Example 5 except that Ca(NO₃)₂.4H₂O was not added, and the composition of the impregnating solution was changed so as to achieve the composition of the catalyst 14 shown in Table 3.

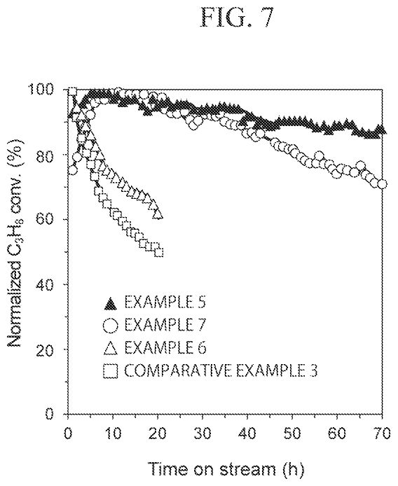

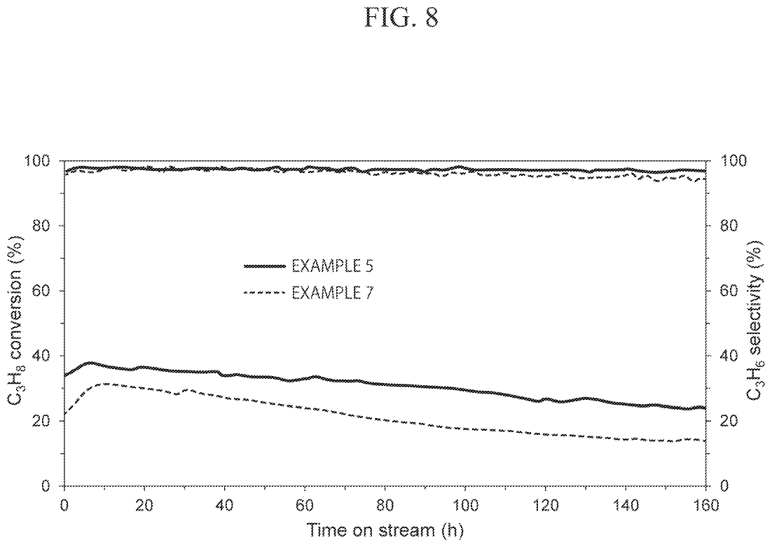

The dehydrogenation reactions of propane were carried out using the catalysts of Examples 5 to 7 and Comparative Example 3. It should be noted that as a reaction condition, the reaction condition 3 shown in Table 1 was employed in Example 6 and Comparative Example 3, and the reaction condition 2 shown in Table 1 was employed in Examples 5 and 7. The changes in the relative conversion rate of propane over time are shown in FIG. 7. It should be noted that the “Normalized CH₃H₈ conv. (Relative conversion rate)” in FIG. 7 is a relative conversion rate when the maximum conversion rate in each catalyst in a reaction time of 0 to 70 hours was taken as 100%. Compared with the catalyst of Comparative Example 3 containing only Pt and Ga, the catalyst of Example 6 containing Pt, Ga and Ca suppressed the decrease in the conversion rate over time. Similarly, the decrease in the conversion rate over time was suppressed in the catalyst of Example 5 containing Pt, Ga, Pb and Ca, as compared with the catalyst of Example 7 containing Pt, Ga and Pb. The changes in the conversion rate of propane over time and the changes in the selectivity of propylene over time in the catalyst of Example 5 and the catalyst of Example 7 are shown in FIG. 8.

### Example 8

A catalyst 15 in which Pt, Ga and Ca were supported on silica was obtained in the same manner as in Example 6 except that the composition of the impregnating solution was changed so as to achieve the composition of the catalyst 15 shown in Table 3.

### Example 91

A catalyst 16 in which Pt, Ga and Ca were supported on silica was obtained in the same manner as in Example 6 except that the composition of the impregnating solution was changed so as to achieve the composition of the catalyst 16 shown in Table 3.

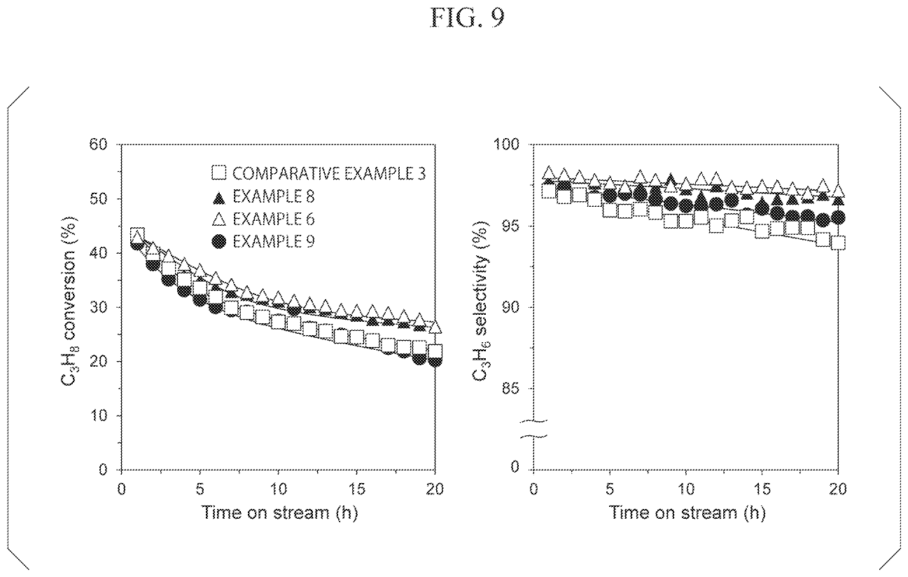

The dehydrogenation reactions of propane were carried out using the catalysts of Examples 6,8 and 9 and Comparative Example 3. It should be noted that the reaction condition 3 shown in Table 1 was employed as a reaction condition. The changes in the conversion rate of propane over time and the changes in the selectivity of propylene over time are shown in FIG. 9. The catalysts of Examples 6, 8 and 9 containing Pt, Ga and Ca had higher yields of propylene at any reaction elapsed time, as compared with the catalyst of Comparative Example 3 containing Pt and Ga. Further, as compared with the catalyst of Example 9 having a Ca/Pt molar ratio of 7, the catalyst of Example 6 having a Ca/Pt molar ratio of 5 and the catalyst of Example 8 having a Ca/Pt molar ratio of 3 suppressed the decrease in propane conversion rate and propylene selectivity.

### Comparative Example 8

A catalyst 17 in which Pt, Ga and Na were supported on silica was obtained in the same manner as in Example 6 except that Ca(NO₃)₂.4H₂O was not added, NaNO₃ was added as a Na source, and the composition of the impregnating solution was changed so as to achieve the composition of the catalyst 17 shown in Table 3. It should be noted that a molar ratio of Pt:Na was 1:5.

### Comparative Example 9

A catalyst 18 in which Pt, Ga and K were supported on silica was obtained in the same manner as in Example 6 except that Ca(NO₃)₂.4H₂O was not added, KNO₃ was added as a K source, and the composition of the impregnating solution was changed so as to achieve the composition of the catalyst 18 shown in Table 3. It should be noted that a molar ratio of Pt:K was 1:5.

### Comparative Example 10

A catalyst 19 in which Pt, Ga and Rb were supported on silica was obtained in the same manner as in Example 6 except that Ca(NO₃)₂.4H₂O was not added, Rb₂CO₃ was added as an Rb source, and the composition of the impregnating solution was changed so as to achieve the composition of the catalyst 19 shown in Table 3. It should be noted that a molar ratio of Pt:Rb was 1:5.

### Comparative Example 11

A catalyst 20 in which Pt, Ga and Cs were supported on silica was obtained in the same manner as in Example 6 except that Ca(NO₃)₂.4H₂O was not added, CsNO₃ was added as a Cs source, and the composition of the impregnating solution was changed so as to achieve the composition of the catalyst 20 shown in Table 3. It should be noted that a molar ratio of Pt:Cs was 1:5.

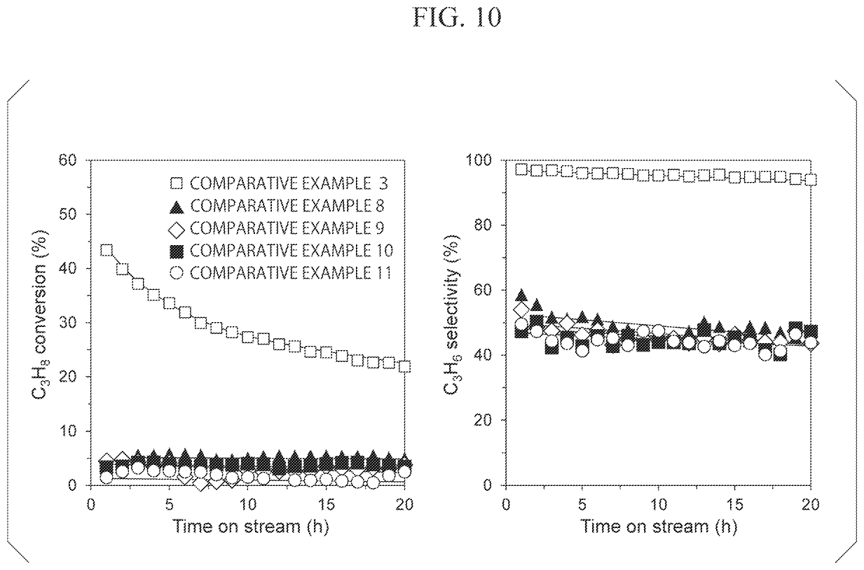

The dehydrogenation reactions of propane were carried out using the catalysts of Comparative Examples 3 and 8 to 11. It should be noted that the reaction condition 3 shown in Table 1 was employed as a reaction condition. The changes in the conversion rate of propane over time and the changes in the selectivity of propylene over time are shown in FIG. 10. The propane conversion rates of the catalysts of Comparative Examples 8 to 11 using alkali metals instead of Ca were extremely low.

### Comparative Example 121

A catalyst 21 in which Pt, Ga and Mg were supported on silica was obtained in the same manner as in Example 6 except that Ca(NO₃)₂.4H₂O was not added, Mg(NO₃)₂.6H₂O was added as a Mg source, and the composition of the impregnating solution was changed so as to achieve the composition of the catalyst 21 shown in Table 3. It should be noted that a molar ratio of Pt:Mg was 1:5.

### Comparative Example 131

A catalyst 22 in which Pt, Ga and Sr were supported on silica was obtained in the same manner as in Example 6 except that Ca(NO₃)₂.4H₂O was not added, Sr(NO₃)₂ was added as an Sr source, and the composition of the impregnating solution was changed so as to achieve the composition of the catalyst 22 shown in Table 3. It should be noted that a molar ratio of Pt:Sr was 1:5.

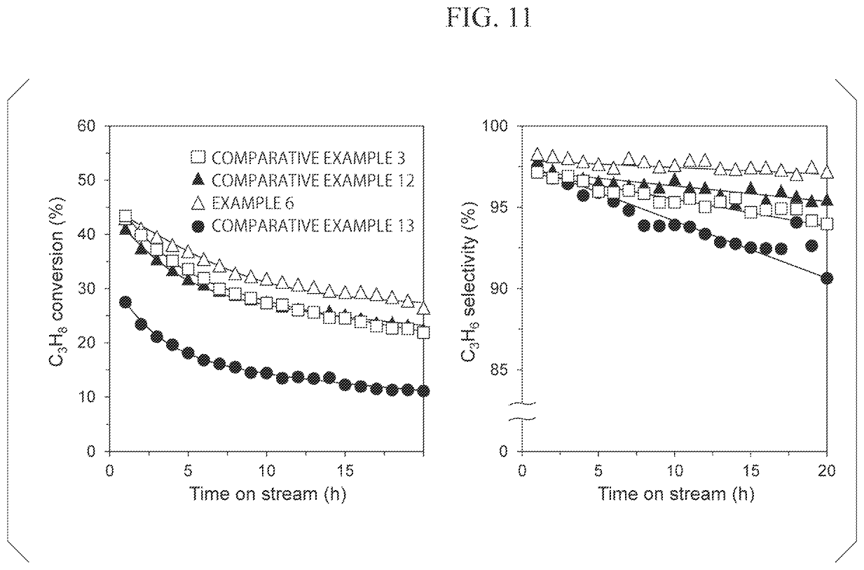

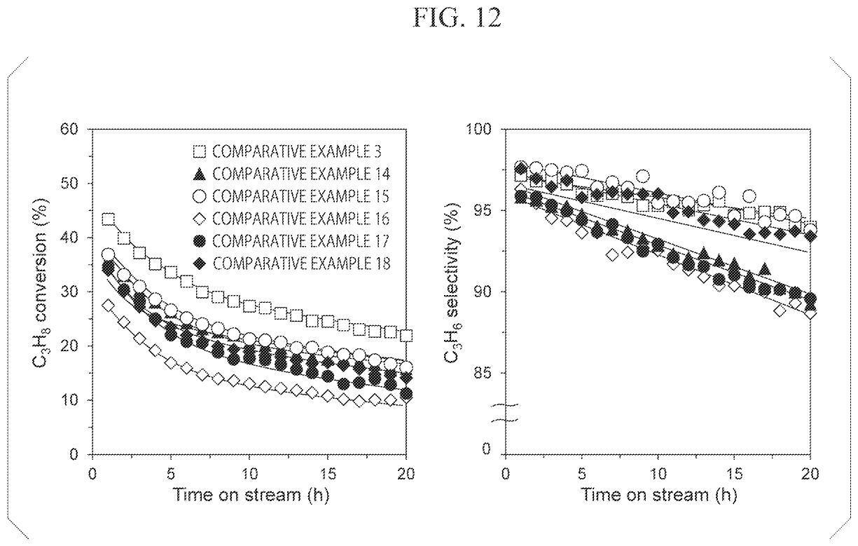

The dehydrogenation reactions of propane were carried out using the catalysts of Example 6 and Comparative Examples 3, 12 and 13. It should be noted that the reaction condition 3 shown in Table 1 was employed as a reaction condition. The changes in the conversion rate of propane over time and the changes in the selectivity of propylene over time are shown in FIG. 11. The propane conversion rate of the catalyst of Comparative Example 13 using Sr, which was also an alkaline earth metal, instead of Ca was extremely low. Further, the catalyst of Comparative Example 12 using Mg, which was also an alkaline earth metal, instead of Ca had a propane conversion rate substantially equivalent to that of the catalyst of Comparative Example 3 containing no Mg, and the effect of suppressing the decrease in propane conversion rate over time was not confirmed.

### Comparative Example 14

A catalyst 23 in which Pt, Ga and Y were supported on silica was obtained in the same manner as in Example 6 except that Ca(NO₃)₂.4H₂O was not added, Y(NO₃)₃.6H₂O was added as a Y source, and the composition of the impregnating solution was changed so as to achieve the composition of the catalyst 23 shown in Table 3. It should be noted that a molar ratio of Pt:Y was 1:5.

### Comparative Example 15

A catalyst 24 in which Pt, Ga and La were supported on silica was obtained in the same manner as in Example 6 except that Ca(NO₃)₂.4H₂O was not added, La(NO₃)₃.6H₂O was added as a La source, and the composition of the impregnating solution was changed so as to achieve the composition of the catalyst 24 shown in Table 3. It should be noted that a molar ratio of Pt:La was 1:5.

### Comparative Example 16

A catalyst 25 in which Pt. Ga and Ce were supported on silica was obtained in the same manner as in Example 6 except that Ca(NO₃)₂.4H₂O was not added, Ce(NO₃)₃.6H₂O was added as a Ce source, and the composition of the impregnating solution was changed so as to achieve the composition of the catalyst 25 shown in Table 3. It should be noted that a molar ratio of Pt:Ce was 1:5.

### Comparative Example 17

A catalyst 26 in which Pt, Ga and Nd were supported on silica was obtained in the same manner as in Example 6 except that Ca(NO₃)₂.4H₂O was not added, Nd(NO₃)₃.6H₂O was added as an Nd source, and the composition of the impregnating solution was changed so as to achieve the composition of the catalyst 26 shown in Table 3. It should be noted that a molar ratio of Pt:Nd was 1:5.

### Comparative Example 18

A catalyst 27 in which Pt, Ga and Sm were supported on silica was obtained in the same manner as in Example 6 except that Ca(NO₃)₂.4H₂O was not added, Sm(NO₃)₃.6H₂O was added as a Sm source, and the composition of the impregnating solution was changed so as to achieve the composition of the catalyst 27 shown in Table 3. It should be noted that a molar ratio of Pt; Sm was 1:5.

The dehydrogenation reactions of propane were carried out using the catalysts of Comparative Examples 3 and 14 to 18. It should be noted that the reaction condition 3 shown in Table 1 was employed as a reaction condition. The changes in the conversion rate of propane over time and the changes in the selectivity of propylene over time are shown in FIG. 12. The propane conversion rates of the catalysts of Comparative Examples 14 to 18 using Y, La, Ce, Nd and Sm instead of Ca were extremely low.

TABLE 3

| | | | | | | | | | | Carbon | |
|---|---|---|---|---|---|---|---|---|---|---|---|
| | | | | | | | | | | content of | |
| | | | | | | | | Element | Average | catalyst | |
| | | | Amount of | | | | | other than | particle | after | Pt |
| | | | supported Pt | Molar ratio | | | | Pt, Ga | size | reaction | dispersity |
| | Catalyst | Carrier | mass % | Pt | Ga | Pb | Ca | and M | nm | g_{coke}/g_{cat} | % |
| Ex. 5 | Catalyst 12 | SiO₂ | 3 | 1 | 1 | 0.55 | 5 | — | 2.2 | 0.0096 | 2.6 |
| Ex. 6 | Catalyst 13 | SiO₂ | 3 | 1 | 1 | 0 | 5 | — | 2.4 | — | 10.6 |
| Ex. 7 | Catalyst 14 | SiO₂ | 3 | 1 | 1 | 0.75 | 0 | — | 2.5 | 0.0190 | 1.0 |
| Ex. 8 | Catalyst 15 | SiO₂ | 3 | 1 | 1 | 0 | 3 | — | — | — | — |
| Ex. 9 | Catalyst 16 | SiO₂ | 3 | 1 | 1 | 0 | 7 | — | — | — | — |
| Comp. Ex. 8 | Catalyst 17 | SiO₂ | 3 | 1 | 1 | 0 | 0 | Na | — | — | — |
| Comp. Ex. 9 | Catalyst 18 | SiO₂ | 3 | 1 | 1 | 0 | 0 | K | — | — | — |
| Comp. Ex. 10 | Catalyst 19 | SiO₂ | 3 | 1 | 1 | 0 | 0 | Rb | — | — | — |
| Comp. Ex. 11 | Catalyst 20 | SiO₂ | 3 | 1 | 1 | 0 | 0 | Cs | — | — | — |
| Comp. Ex. 12 | Catalyst 21 | SiO₂ | 3 | 1 | 1 | 0 | 0 | Mg | — | — | — |
| Comp. Ex. 13 | Catalyst 22 | SiO₂ | 3 | 1 | 1 | 0 | 0 | Sr | — | — | — |
| Comp. Ex. 14 | Catalyst 23 | SiO₂ | 3 | 1 | 1 | 0 | 0 | Y | — | — | — |
| Comp. Ex. 15 | Catalyst 24 | SiO₂ | 3 | 1 | 1 | 0 | 0 | La | — | — | — |
| Comp. Ex. 16 | Catalyst 25 | SiO₂ | 3 | 1 | 1 | 0 | 0 | Ce | — | — | — |
| Comp. Ex. 17 | Catalyst 26 | SiO₂ | 3 | 1 | 1 | 0 | 0 | Nd | — | — | — |
| Comp. Ex. 18 | Catalyst 27 | SiO₂ | 3 | 1 | 1 | 0 | 0 | Sm | — | — | — |

### Comparative Example 191

Magnesia was impregnated with an impregnating solution prepared by dissolving Pt(NH₃)₂(NO₃) as a Pt source and Ga(NO₃)₃.nH₂O (n=7 to 9) as a Ga source in ion-exchanged water so as to achieve the composition of the catalyst 28 shown in Table 4 by a method of evaporation to dryness. The obtained impregnated body was dried under the conditions of 50° C. and reduced pressure. The obtained powder was further dried overnight in an oven at 90° C., followed by calcining at 600° C. for 1 hour in an air atmosphere, and then subjected to reduction calcining at 700° C. for 1 hour while circulating hydrogen (0.1 MPa, 50 NmL/min) to obtain a catalyst 28 in which Pt and Ga were supported on magnesia.

### Comparative Example 20

A catalyst 29 shown in Table 4 was obtained in the same manner as in Comparative Example 19 except that ceria was used instead of magnesia as a carrier.

### Comparative Example 21

A catalyst 30 shown in Table 4 was obtained in the same manner as in Comparative Example 19 except that zirconia was used instead of magnesia as a carrier.

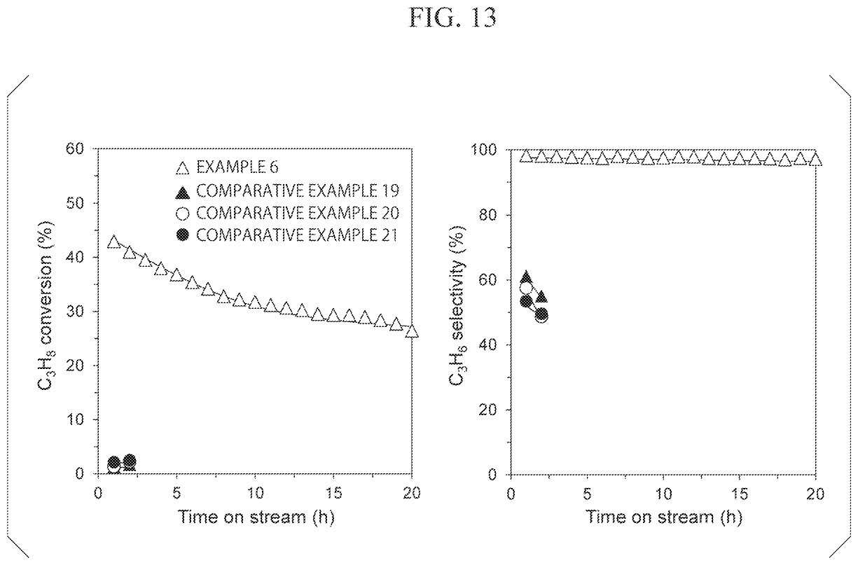

The dehydrogenation reactions of propane were carried out using the catalysts of Example 6 and Comparative Examples 19 to 21. It should be noted that the reaction condition 3 shown in Table 1 was employed as a reaction condition. The changes in the conversion rate of propane over time and the changes in the selectivity of propylene over time are shown in FIG. 13. The propane conversion rates of the catalysts of Comparative Examples 19 to 21 were extremely low.

### Comparative Example 22

A catalyst 31 shown in Table 4 was obtained in the same manner as in Comparative Example 19 except that alumina was used instead of magnesia as a carrier.

### Comparative Example 231

A catalyst 32 shown in Table 4 was obtained in the same manner as in Comparative Example 19 except that titania was used instead of magnesia as a carrier.

### Comparative Example 24

A catalyst 33 shown in Table 4 was obtained in the same manner as in Comparative Example 19 except that MgAl₂O₄ was used instead of magnesia as a carrier.

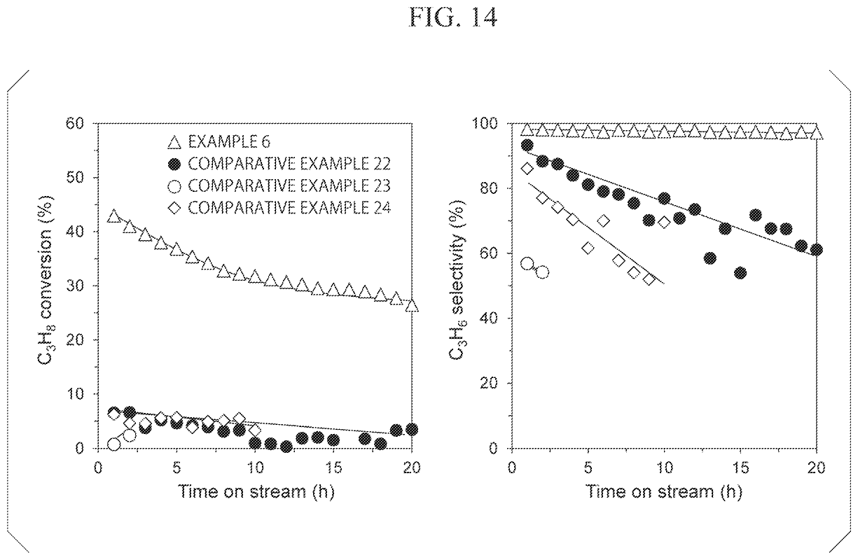

The dehydrogenation reactions of propane were carried out using the catalysts of Example 6 and Comparative Examples 22 to 24. It should be noted that the reaction condition 3 shown in Table 1 was employed as a reaction condition. The changes in the conversion rate of propane over time and the changes in the selectivity of propylene over time are shown in FIG. 14. The propane conversion rates of the catalysts of Comparative Examples 22 to 24 were extremely low.

TABLE 4

| | | | Amount of | | |
|---|---|---|---|---|---|
| | | | supported Pt | Molar ratio | |
| | Catalyst | Carrier | mass % | Pt | Ga |
| Comp. Ex. 19 | Catalyst 28 | MgO | 3 | 1 | 1 |
| Comp. Ex. 20 | Catalyst 29 | CeO₂ | 3 | 1 | 1 |
| Comp. Ex. 21 | Catalyst 30 | ZrO₂ | 3 | 1 | 1 |
| Comp. Ex. 22 | Catalyst 31 | Al₂O₃ | 3 | 1 | 1 |
| Comp. Ex. 23 | Catalyst 32 | TiO₂ | 3 | 1 | 1 |
| Comp. Ex. 24 | Catalyst 33 | MgAl₂O₄ | 3 | 1 | 1 |

### Example 10

Silica (product name “CARiACT G-6”, manufactured by Fuji Silysia Chemical Ltd., specific surface area: 673 m²/g) was impregnated with an impregnating solution prepared by dissolving Pt(NH₃)₂(NO₃)₂ as a Pt source, Co(NO₃)₂.6H₂O as a Co source, Cu(NO₃)₂.3H₂O as a Cu source, Ga(NO₃)₃.nH₂O (n=7 to 9) as a Ga source, (NH₄)₂GeF₆ as a Ge source, (NH₄)₂SnCl₂ as a Sn source and Ca(NO₃)₂.4H₂O as a Ca source in ion-exchanged water so as to achieve the composition of the catalyst 34 shown in Table 5 by a pore filling method. The obtained impregnated body was stored overnight at room temperature in a sealed round bottom flask, and subsequently frozen with liquid nitrogen and freeze dried in vacuum at −5° C. The obtained powder was further dried overnight in an oven at 90° C., followed by calcining at 400° C. for 1 hour in an air atmosphere, and then subjected to reduction calcining at 700° C. for 1 hour while circulating hydrogen (0.1 MPa, 50 NmL/min) to obtain a catalyst 34 in which Pt, Co, Cu, Ga, Ge, Sn and Ca were supported on silica.

### Comparative Example 251

Silica (product name “CARiACT G-6”, manufactured by Fuji Silysia Chemical Ltd., specific surface area: 673 m²/g) was impregnated with an impregnating solution prepared by dissolving Pt(NH₃)₂(NO₃)₂ as a Pt source, (NH₄)₂GeF₆ as a Ge source and Ca(NO₃)₂.4H₂O as a Ca source in ion-exchanged water so as to achieve the composition of the catalyst 35 shown in Table 5 by a pore filling method. The obtained impregnated body was stored overnight at room temperature in a sealed round bottom flask, and subsequently frozen with liquid nitrogen and freeze dried in vacuum at −5° C. The obtained powder was further dried overnight in an oven at 90° C., followed by calcining at 300° C. for 1 hour in an air atmosphere, and then subjected to reduction calcining at 700° C. for 1 hour while circulating hydrogen (0.1 MPa, 50 NmL/min) to obtain a catalyst 35 in which Pt, Ge and Ca were supported on silica.

### Example 11

Silica (product name “CARiACT G-6”, manufactured by Fuji Silysia Chemical Ltd., specific surface area: 673 m²/g) was impregnated with an impregnating solution prepared by dissolving Pt(NH₃)₂(NO₃)₂ as a Pt source, Co(NO₃)₂.6H₂O as a Co source, Cu(NO₃)₂.3H₂O as a Cu source, Ga(NO₃)₃.nH₂O (n=7 to 9) as a Ga source, Fe(NO₃)₂.6H₂O as a Fe source and Ca(NO₃)₂.4H₂O as a Ca source in ion-exchanged water so as to achieve the composition of the catalyst 36 shown in Table 5 by a pore filling method. The obtained impregnated body was stored overnight at room temperature in a sealed round bottom flask, and subsequently frozen with liquid nitrogen and freeze dried in vacuum at −5° C. The obtained powder was further dried overnight in an oven at 90° C., followed by calcining at 400° C. for 1 hour in an air atmosphere, and then subjected to reduction calcining at 700° C. for 1 hour while circulating hydrogen (0.1 MPa, 50 NmL/min) to obtain a catalyst 36 in which Pt, Co, Cu, Ga, Fe and Ca were supported on silica.

### Example 12

Silica (product name “CARiACT G-6”, manufactured by Fuji Silysia Chemical Ltd., specific surface area: 673 m²/g) was impregnated with an impregnating solution prepared by dissolving Pt(NH₃)₂(NO₃)₂ as a Pt source, Cu(NO₃)₂.3H₂O as a Cu source and Ca(NO₃)₂.4H₂O as a Ca source in ion-exchanged water so as to achieve the composition of the catalyst 37 shown in Table 5 by a pore filling method. The obtained impregnated body was stored overnight at room temperature in a sealed round bottom flask, and subsequently frozen with liquid nitrogen and freeze dried in vacuum at −5° C. The obtained powder was further dried overnight in an oven at 90° C., followed by calcining at 400° C. for 1 hour in an air atmosphere, and then subjected to reduction calcining at 700° C. for 1 hour while circulating hydrogen (0.1 MPa, 50 NmL/min) to obtain a catalyst 37 in which Pt, Cu and Ca were supported on silica.

### Example 13

A catalyst 38 in which Pt, Co, Cu, Ga, Ge, Sn and Ca were supported on silica was obtained in the same manner as in Example 10 except that the composition of the impregnating solution was changed so as to achieve the composition of the catalyst 38 shown in Table 5.

### Example 14

A catalyst 39 in which Pt, Co, Cu, Ga, Ge, Sn and Ca were supported on silica was obtained in the same manner as in Example 10 except that the composition of the impregnating solution was changed so as to achieve the composition of the catalyst 39 shown in Table 5.

TABLE 5

| | | | | | | | | | | | | | Carbon |
|---|---|---|---|---|---|---|---|---|---|---|---|---|---|
| | | | | | | | | | | | | | content of |
| | | | | | | | | | | | | Average | catalyst |
| | | | Amount of | | | | | | | | | particle | after |
| | | | supported Pt | Molar ratio | | | | | | | | size | reaction |
| | Catalyst | Carrier | mass % | Pt | Co | Cu | Ga | Ge | Sn | Fe | Ca | nm | g_{coke}/g_{cat} |
| Ex. 10 | Catalyst 34 | SiO₂ | 1 | 1 | 1.5 | 1.5 | 2 | 1.5 | 1.5 | 0 | 15 | 2.2 | 0.016 |
| Comp. Ex. 25 | Catalyst 35 | SiO₂ | 1 | 1 | 0 | 0 | 0 | 2 | 0 | 0 | 15 | 1.8 | 0.042 |
| Ex. 11 | Catalyst 36 | SiO₂ | 1 | 1 | 1.5 | 1.5 | 3 | 0 | 0 | 1.5 | 15 | 2.4 | 0.040 |
| Ex. 12 | Catalyst 37 | SiO₂ | 1 | 1 | 0 | 25 | 0 | 0 | 0 | 0 | 15 | 1.9 | 0.021 |
| Ex. 13 | Catalyst 38 | SiO₂ | 1.5 | 1.5 | 1.25 | 1.25 | 2 | 1.5 | 1.5 | 0 | 15 | — | — |
| Ex. 14 | Catalyst 39 | SiO₂ | 2 | 2 | 1 | 1 | 2 | 1.5 | 1.5 | 0 | 15 | — | — |

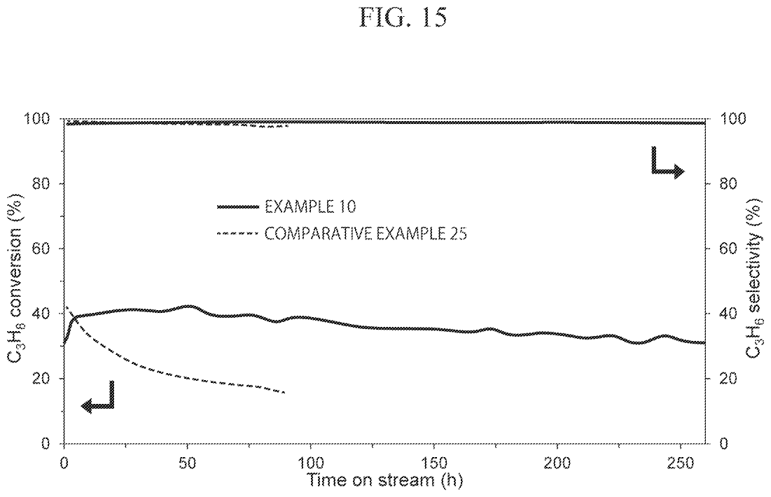

The dehydrogenation reactions of propane were carried out using the catalysts of Example 10 and Comparative Example 25. It should be noted that as a reaction condition, the reaction condition 4 shown in Table 1 was employed for the catalyst of Example 10, and the reaction condition 5 shown in Table 1 was employed for the catalyst of Comparative Example 25. The changes in the conversion rate of propane over time and the changes in the selectivity of propylene over time are shown in FIG. 15. The catalyst of Example 10 containing Pt, Co, Cu, Ga, Ge, Sn and Ca suppressed the decrease in the propane conversion rate over time, and the propane conversion rate hardly decreased even after 250 hours.

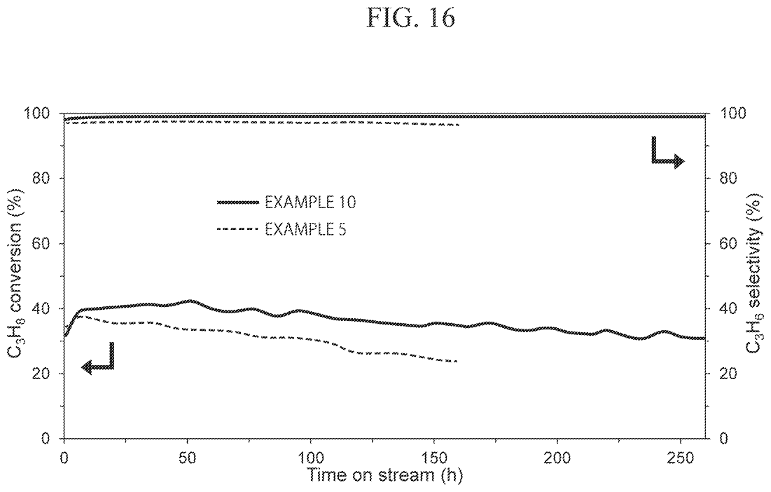

The dehydrogenation reactions of propane were carried out using the catalysts of Examples 10 and 5. It should be noted that as a reaction condition, the reaction condition 4 shown in Table 1 was employed for the catalyst of Example 10, and the reaction condition 2 shown in Table 1 was employed for the catalyst of Example 5. The changes in the conversion rate of propane over time and the changes in the selectivity of propylene over time are shown in FIG. 16. Both the catalyst of Example 5 containing Pt, Ga, Pb and Ca and the catalyst of Example 10 containing Pt, Co, Cu, Ga, Ge, Sn and Ca suppressed the decrease in propane conversion rate over time, and the propane conversion rate hardly decreased even after 250 hours. In particular, a remarkable effect of suppressing the decrease in the propane conversion rate over time was confirmed for the catalyst of Example 10.

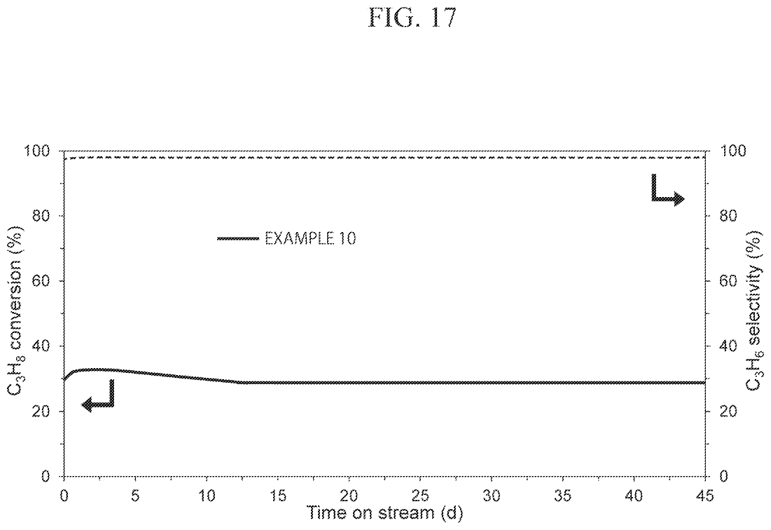

A life test was carried out in the dehydrogenation reaction of propane using the catalyst of Example 10. It should be noted that the reaction condition 6 shown in Table 1 was employed as a reaction condition. The changes in the conversion rate of propane over time and the changes in the selectivity of propylene over time are shown in FIG. 17. The catalyst of Example 10 containing Pt, Co, Cu, Ga, Ge, Sn and Ca suppressed the decrease in the propane conversion rate over time, and the propane conversion rate hardly decreased even after 45 days.

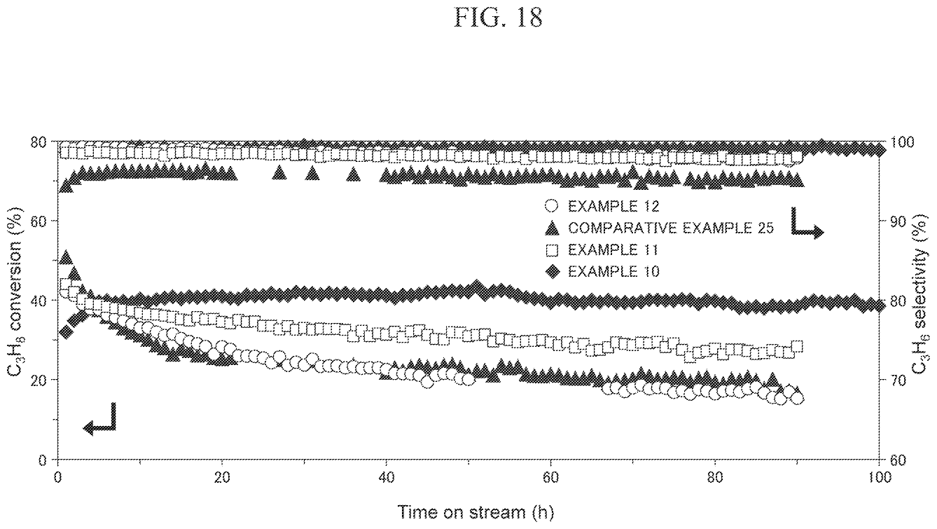

The dehydrogenation reactions of propane were carried out using the catalysts of Examples 10 to 12 and Comparative Example 25. It should be noted that as a reaction condition, the reaction condition 4 shown in Table 1 was employed for the catalyst of Example 10, the reaction condition 8 shown in Table 1 was employed for the catalyst of Example 11, the reaction condition 4 shown in Table 1 was employed for the catalyst of Example 12, and the reaction condition 5 shown in Table 1 was employed for the catalyst of Comparative Example 25. The changes in the conversion rate of propane over time and the changes in the selectivity of propylene over time are shown in FIG. 18. It was found that both the conversion rate of propane and the selectivity of propylene were extremely high for the catalyst of Example 10 and the catalyst of Example 11, as compared with those of the catalyst of Comparative Example 25. Further, it was found that the catalyst of Example 12 had a higher selectivity of propylene, as compared with the catalyst of Comparative Example 25.

The dehydrogenation reactions of propane were carried out using the catalysts of Examples 10, 11, 13 and 14 and Comparative Example 25. It should be noted that as a reaction condition, the reaction condition 4 shown in Table 1 was employed for the catalyst of Example 10, the reaction condition 7 shown in Table 1 was employed for the catalyst of Example 13, the reaction condition 5 shown in Table 1 was employed for the catalyst of Example 14, the reaction condition 8 shown in Table 1 was employed for the catalyst of Example 11, and the reaction condition 5 shown in Table 1 was employed for the catalyst of Comparative Example 25. Table 6 shows the average catalyst life t calculated by the aforementioned equations (3) and (4). It was found that the catalyst of Example 10, the catalyst of Example 11, the catalyst of Example 13 and the catalyst of Example 14 had an extremely long average catalyst life, as compared with the catalyst of Comparative Example 25. Although the catalyst of Example 10, the catalyst of Example 13 and the catalyst of Example 14 contain the same types of elements, the aforementioned Pt/(transition metal group 1) ratio decreases in the order of the catalyst of Example 14, the catalyst of Example 13 and the catalyst of Example 10. It was found that the catalyst of Example 10 having the lowest Pt/(transition metal group 1) ratio of 0.25 had a particularly long average catalyst life.

TABLE 6

| | | t (h) | | C₃H₈ conversion (%) | | C₃H₆ selectivity (%) | | | |
|---|---|---|---|---|---|---|---|---|---|
| | Catalyst | start | end | start | end | start | end | K_{d} (h⁻¹) | τ (h) |
| Ex. 10 | Catalyst 34 | 10 | 260 | 40.3 | 31.2 | 99.3 | 98.7 | 0.002 | 628 |
| Comp. Ex. 25 | Catalyst 35 | 1 | 90 | 42.1 | 15.3 | 99.3 | 97.9 | 0.016 | 64 |
| Ex. 11 | Catalyst 36 | 1 | 90 | 44.0 | 28.3 | 98.6 | 98.0 | 0.008 | 130 |
| Ex. 13 | Catalyst 38 | 10 | 119 | 32.2 | 27.7 | 98.6 | 98.4 | 0.002 | 509 |
| Ex. 14 | Catalyst 39 | 10 | 120 | 39.4 | 32.1 | 98.7 | 98.9 | 0.003 | 343 |

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

### INDUSTRIAL APPLICABILITY

The dehydrogenation catalyst according to the present invention is useful because propylene can be produced for a long period of time.

What is claimed is:

1. A dehydrogenation catalyst for producing propylene by a dehydrogenation reaction of propane, the dehydrogenation catalyst comprising:
a platinum element and an element M1, and may contain an element M2 as active components,
wherein said element M1 is one or more elements selected from the group consisting of a gallium element, a cobalt element, a copper element, a germanium element, a tin element and an iron element,
said element M2 is one or more elements selected from the group consisting of a lead element and a calcium element, and
said platinum element and said element M1 form an alloy (provided that a dehydrogenation catalyst containing only a tin element as the element M1 and not containing an element M2, a dehydrogenation catalyst containing only a gallium element as the element M1 and not containing an element M2, a dehydrogenation catalyst containing only a cobalt element as the element M1 and not containing an element M2, a dehydrogenation catalyst containing only a copper element as the element M1 and not containing an element M2, a dehydrogenation catalyst containing only a germanium element as the element M1 and not containing an element M2, and a dehydrogenation catalyst containing only a germanium element as the element M1 and containing only a calcium element as the element M2 are excluded).

2. The dehydrogenation catalyst according to claim 1, which comprises a gallium element as said element M1.

3. The dehydrogenation catalyst according to claim 2, which comprises a lead element as said element M2.

4. The dehydrogenation catalyst according to claim 3, wherein said lead element is present as an atom on a surface of said alloy.

5. The dehydrogenation catalyst according to claim 2, which comprises a cobalt element, a copper element, a germanium element and a tin element as said element M1.

6. The dehydrogenation catalyst according to claim 2, which comprises a cobalt element, a copper element and an iron element as said element M1.

7. The dehydrogenation catalyst according to claim 1, which comprises a copper element as said element M1.

8. The dehydrogenation catalyst according to claim 1, which comprises a calcium element as said element M2.

9. The dehydrogenation catalyst according to claim 1, wherein said active component is supported on a silica carrier.
